SEQUENCE LISTING (10 sequences)

SEQ ID NO: 1 - protein, 497 aa, Oryza sativa
MEGVGGGGGGEEWLRPMDAEQLRECGHRMVDFVADYYKSIEAFPVLSQVQPGYLKEVLPDSAPRQPDTLDSLFDDIQQKIIPGVTHWQSPNYFAYYPSNSSTAGFLGEMLSAAFNIVGFSWITSPAATELEVIVLDWFAKMLQLPSQFLSTALGGGVIQGTASEAVLVALLAARDRALKKHGKHSLEKLVVYASDQTHSALQKACQIAGIFSENVRVVIADCNKNYAVAPEAVSEALSIDLSSGLIPFFICATVGTTSSSAVDPLPELGQIAKSNDMWFHIDAAYAGSACICPEYRHHLNGVEEADSFNMNAHKWFLTNFDCSLLWVKDRSFLIQSLSTNPEFLKNKASQANSVVDFKDWQIPLGRRFRSLKLWMVLRLYGVDNLQSYIRKHIHLAEHFEQLLLSDSRFEVVTPRTFSLVCFRLVPPTSDHENGRKLNYDMMDGVNSSGKIFLSHTVLSGKFVLRFAVGAPLTEERHVDAAWKLLRDEATKVLGKMV

SEQ ID NO: 2 - protein, 575 aa, Modestobacter marinus
MTGHMTPEQFRQHGHEVVDWIADYWERIGSFPVRSQVSPGDVRASLPPTAPEQGEPFSAVLADLDRVVLPGVTHWQHPGFFGYFPANTSGPSVLGDLVSAGLGVQGMSWVTSPAATELEQHVMDWFADLLGLPESFRSTGSGGGVVQDSSSGANLVALLAALHRASKGATLRHGVRPEDHTVYVSAETHSSMEKAARIAGLGTDAIRIVEVGPDLAMNPRALAQRLERDVARGYTPVLVCATVGTTSTTAIDPLAELGPICQQHGVWLHVDAAYAGVSAVAPELRALQAGVEWADSYTTDAHKWLLTGFDATLFWVADRAALTGALSILPEYLRNAATDTGAVVDYRDWQIELGRRFRALKLWFVVRWYGAEGLREHVRSHVALAQELAGWADADERFDVAAPHPFSLVCLRPRWAPGIDADVATMTLLDRLNDGGEVFLTHTTVDGAAVLRVAIGAPATTREHVERVWALLGEAHDWLARDFEEQAAERRAAELREREAAEEQLRARREAEAAAAAATEAPVEPAAEEPEQLVVPPVEVPAVETPAAWDESATQVAAQTDLHADPAPQPADGQG

SEQ ID NO: 3 - protein, 481 aa, Streptomyces sviceus
MPDLEPDEFRRQGHQLVDWVARYRTSLPSLHVRPKVVPGSVKAQLPRELPEQPSQALGDDLIALLNDVVVPSSLHWQHPGFFGYFPANASLLSLLGDIASGGIGAQGMLWSTSPAGTEIEQVLLDGLADALGLGREFTFAGGGGGSLQDSASSASLAALLAALQRSNPDWREHGVDGTETVYVTAETHSSLAKAVRVAGLGARALRIVPFTQGTLSMSADALADMLAKDTAAGKRPVMVCPTVGTTGTGAIDPVREVALAARTYEAWVHVDAAWAGVAALCPEFRWLLDGVNLVDSFCTDAHKWFYTAFDASFMWVRDARALPTALSITPEYLRNAATESGEVIDYRDWQVPLGRRMRALKIWSVVHGAGLEGLRESIRGHVAMANSLAGRIESESGFALATPPSLALVCLYLVDQEGRPDDAATKAAMEAVNAEGHSFLTHTSVNGHFAIRVAIGATTTLPDHIDTLWDSLCKAARQSGG

SEQ ID NO: 4 - protein, 470 aa, Pseudomonas putida
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKG

SEQ ID NO: 5 - protein, 384 aa, Propionibacterium sp.
MGMDISSRPVEWASLSEITASDVSFEGGAIFNSICTRPHPLAAQVMADNLHLNAGDGRLFPSVARCESEITNFLGGLMGLPRAVGMCTSGATEANLIAVHSAIENWRRKGGQGRPQVILGRGGHFSFDKISVLLGVELVLAWSDIDTLKVDPESVSELISPRTALIVATAGSSETGAVDDVEWLSRVALSKGVPLHVDAASGGLLIPFLRDLGGALPDIGFRNDGVTTIAIDPHKFGSAPIPSGHLVAREWTWIEGLRTESHYQGTARHLTFLGTRSGGSILATYALFGHLGEKGLRGMAEQLKALRSHLVDRLRKAGATLAYVPELMVVALKADSDAVKVLERRGIFTSYAKRLGYLRIVVQLHMSEGQVDGLVDALLMEGIV

SEQ ID NO: 6 - protein, 361 aa, Enterococcus faecium
TKLQNNELKRGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLKDLSFKVGTKNRSSSITTH

SEQ ID NO: 7 - protein, 363 aa, Methanosphaerula palustris
MLNKGLAEEELFSFLSKKREEDLCHSHILSSMCTVPHPIAVKAHLMFMETNLGDPGLFPGTASLERLLIERLGDLFHHREAGGYATSGGTESNIQALRIAKAQKKVDKPNVVIPETSHFSFKKACDILGIQMKTVPADRSMRTDISEVSDAIDKNTIALVGIAGSTEYGMVDDIGALATIAEEEDLYLHVDAAFGGLVIPFLPNPPAFDFALPGVSSIAVDPHKMGMSTLPAGALLVREPQMLGLLNIDTPYLTVKQEYTLAGTRPGASVAGALAVLDYMGRDGMEAVVAGCMKNTSRLIRGMETLGFPRAVTPDVNVATFITNHPAPKNWVVSQTRRGHMRIICMPHVTADMIEQFLIDIGE

SEQ ID NO: 8 - protein, 432 aa, Petroselinum crispum
EFRRQGHLMIDFLADYYRKVENYPVRSQVSPGYLREILPESAPYNPESLETILQDVQTKIIPGITHWQSPNFFAYFPSSGSTAGFLGEMLSTGFNVVGFNWMVSPAATELENVVTDWFGKMLQLPKSFLFSGGGGGVLQGTTCEAILCTLVAARDKNLRQHGMDNIGKLVVYCSDQTHSALQKAAKIAGIDPKNFRAIETSKSSNFKLCPKRLESAILYDLQNGLIPLYLCATVGTTSSTTVDPLPALTEVAKKYKLWVHVDAAYAGSACICPEFRQYLDGVENADSFSLNAHKWFLTTLDCCCLWVRDPSALIKSLSTYPEFLKNNASETNKVVDYKDWQIMLSRRFRALKLWFVLRSYGVGQLREFIRGHVGMAKYFEGLVGMDNRFEVVAPRLFSMVCFRIKPSAMIGKNDEDEVNEINRKLLESVNDS

SEQ ID NO: 9 - protein, 396 aa, Methanocaldococcus jannaschii
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 10 - protein, 531 aa, Papaver somniferum
MGSLNTEDVLENSSAFGVTNPLDPEEFRRQGHMIIDFLADYYRDVEKYPVRSQVEPGYLRKRLPETAPYNPESIETILQDVTTEIIPGLTHWQSPNYYAYFPSSGSVAGFLGEMLSTGFNVVGFNWMSSPAATELESVVMDWFGKMLNLPESFLFSGSGGGVLQGTSCEAILCTLTAARDRKLNKIGREHIGRLVVYGSDQTHCALQKAAQVAGINPKNFRAIKTFKENSFGLSAATLREVILEDIEAGLIPLFVCPTVGTTSSTAVDPISPICEVAKEYEMWVHVDAAYAGSACICPEFRHFIDGVEEADSFSLNAHKWFFTTLDCCCLWVKDPSALVKALSTNPEYLRNKATESRQVVDYKDWQIALSRRFRSLKLWMVLRSYGVTNLRNFLRSHVKMAKTFEGLICMDGRFEITVPRTFAMVCFRLLPPKTIKVYDNGVHQNGNGVVPLRDENENLVLANKLNQVYLETVNATGSVYMTHAVVGGVYMIRFAVGSTLTEERHVIYAWKILQEHADLILGKFSEADFSS

(19) United States
(12) Patent Application Publication
(10) Pub. No.: US 2021/0225455 A1
(43) Pub. Date: Jul. 22, 2021

(54) BIOREACHABLE PREDICTION TOOL WITH BIOLOGICAL SEQUENCE SELECTION

(71) Applicant: Zymergen Inc., Emeryville, CA (US)

(72) Inventors: Anupam Chowdhury, Oakland, CA (US); Alexander Glennon Shearer, San Francisco, CA (US); Stepan Tymoshenko, Emeryville, CA (US); Michelle L. Wynn, Alameda, CA (US); Erik Jedediah Dean, Lafayette, CA (US)

(73) Assignee: Zymergen Inc., Emeryville, CA (US)

(21) Appl. No.: 17/267,648

(22) PCT Filed: Aug. 14, 2019
(86) PCT No.: PCT/US2019/046580

§ 371 Date: Feb. 10, 2021

Related U.S. Application Data
(60) Provisional application No. 62/720,839, filed on Aug. 21, 2018.
(60) Provisional application No. 62/720,811, filed on Aug. 21, 2018.
(60) Provisional application No. 62/764,819, filed on Aug. 15, 2018.
(60) Provisional application No. 62/764,861, filed on Aug. 15, 2018.

(51) Int. Cl. G16B 5/20; G16B 50/10; G16B 40/30; G16B 40/20; G16B 30/10

(52) U.S. Cl. CPC G16B 5/20; G16B 50/10; G16B 30/10; G16B 40/20; G16B 40/30

(57) ABSTRACT

Systems, methods and non-transitory computer-readable media identify a candidate biological sequence for enabling a function in a host cell. Embodiments access a predictive model that associates a plurality of biological sequences, such as enzymes, with one or more functions, such as reaction catalysis; predict, using the predictive model, that one or more candidate sequences of the plurality of biological sequences enable a desired function; and classify using a processor, candidate sequences that satisfy a confidence threshold as filtered candidate sequences.

### CROSS-REFERENCE TO RELATED APPLICATIONS

This application claims the benefit of U.S. Provisional Application No. 62/764,819, filed Aug. 15, 2018, U.S. Provisional Application No. 62/720,811, filed Aug. 21, 2018, U.S. Provisional Application No. 62/764,861, filed Aug. 15, 2018, and U.S. Provisional Application No. 62/720,839, filed Aug. 21, 2018, all of which are incorporated in their entirety herein.

This application is related to P.C.T. Application No. PCT/US2018/018234, filed on Feb. 14, 2018 (the “BPT PCT application”), and to U.S. Provisional Application No. 62/459,558, filed Feb. 15, 2017, which are incorporated by reference in their entirety herein.

### GOVERNMENT INTEREST STATEMENT

This invention was made with United States Government support under Agreement No. HR0011-15-9-0014, awarded by DARPA. The Government has certain rights in the invention.

### SEQUENCE LISTING

The instant application contains a Sequence Listing which has been submitted electronically in ASCII format and is hereby incorporated by reference in its entirety. Said ASCII copy, created on Aug. 12, 2019, is named ZYM011WOPC01_SL.txt and is 39,253 bytes in size.

### FIELD

The disclosure relates generally to methods which improve genetic engineering of cells and, in particular, to methods which identify molecules that can be produced in a particular cell using an algorithmically selected set of native or heterologous proteins (e.g., enzymes) or gene sequences.

### BACKGROUND

Biologists, chemists, material scientists, and others in related disciplines employ bioengineering to produce desired molecules with desired phenotypic characteristics from cells by, for example, modifying the cell's genome. Such cells may themselves be unicellular organisms (e.g., bacteria) or components of or multicellular host organisms, or may be mutated variants of cells found in nature. However, there is a limit to which molecules can be produced as part of the biomass in a cell. Generally, one is faced with the problem of determining the largest possible pool of bioreachable molecules that may be generated through genetic modification without requiring extensive manual intervention. This problem was addressed in the BPT PCT application.

Embodiments described herein and in the BPT PCT application may a identify candidate bioreachable molecule and a set of reactions leading to its formation. Thereafter, however, the process to engineer a cell to make the molecule typically requires altering the metabolism of the host cell by inserting, deleting, or regulating one or more genes that correspond to an enzymatic catalytic function of a given reaction or reactions. Selection of protein sequences (e.g., enzymes) that have the necessary function, or underlying DNA sequences for coding those protein sequences, from the multitude of all their known and predicted variants is often a hard-to-scale, error-prone process.

Current approaches, such as the RAVEN Toolbox, predict whether desired enzymatic activity is present in a specified genome of interest. The tool box is designed to predict a set of metabolic activities present in a genome with the aim to reconstruct a genome-scale model. However, it is limited to identifying enzymes native to a single host cell for individual reaction activity. Alternative approaches, such as Selenzyme do not include scoring of the candidate enzymes based on the confidence that they have necessary/desired functions or a rational sampling of their sequence diversity as described herein.

Embodiments of the bioreachable prediction tool described in the BPT PCT Application predict bioreachable molecules and reaction pathways to attain those predicted molecules. A chemist or other scientist may use their knowledge and intuition to manually select the optimal enzyme candidates for catalyzing reactions along those pathways. However, the BPT of such embodiments may predict a large number of pathways, each containing multiple reactions (e.g., 10 pathways each containing 10 reactions, or even more), for which manual determination of optimal enzymes is time-consuming and error-prone. Moreover, manual annotation of enzymes can be erroneous, and in other cases may not cause the catalyzed reaction product to be expressed to a desired degree.

### SUMMARY OF THE DISCLOSURE

Embodiments of the disclosure provide a bioreachable prediction tool for predicting viable target molecules and reaction catalysts in a manner that overcomes the disadvantages of conventional techniques. In particular, the bioreachable prediction tool of embodiments of the disclosure predicts viable target molecules that are specific to a host cell and a set of enzymes (which may be native and heterologous) that can be expressed in a given host to enable or improve the production of the molecule.

For each identified reaction pathway (i.e. pedigree) that the bioreachable prediction tool identifies as leading to a viable target molecule, the tool may also identify a set of candidate native or heterologous enzymes for catalyzing each reaction in the reaction pathway idenfied by the BPT, according to embodiments of the disclosure. Embodiments of the disclosure provide a scalable, algorithmic approach that enables rational sampling of the multitude of potential candidate sequences for enabling a given function.

The tool may identify a set of candidate enzymes for catalyzing a particular reaction based at least on one or both of the following: 1) there is evidence the enzymes catalyze a particular targeted reaction, or 2) their sequences match the model for a desired function significantly better than any other models related to the functions other than the desired one.

In embodiments, the tool may further refine the selected set of candidate enzymes for catalysis of a particular reaction based on one or both of the following refinements: 1) there is evidence that the enzymes do not induce additional non-desired functional behaviors in a particular cell, or 2) a model predicted with high probability that the enzymes do not induce other non-desired functional behaviors in a particular host (where non-desired functional behaviors may include, but are not limited to, the catalysis of non-targeted reactions).

Each enzyme in the set of candidate native or heterologous enzymes may then be engineered into one or more host cells in order to catalyze each reaction in a particular reaction pathway (pedigree) identified as able to produce the desired target molecule. In embodiments, the tool may also ensure that the identified set of candidate enzymes are evolutionary diverse from one another, while still maintaining confidence in the presence of the required catalytic activity.

Embodiments of the disclosure provide systems, methods and non-transitory computer-readable media for identifying a candidate biological sequence for enabling a function in a host cell. Embodiments access a predictive model that associates a plurality of biological sequences with one or more functions; predict, using the predictive model, that one or more candidate sequences of the plurality of biological sequences enable a desired function; and classify using a processor, candidate sequences that satisfy a confidence threshold as filtered candidate sequences. Processing of a first filtered candidate sequence of the filtered candidate sequences may result in production of a molecule. Embodiments of the disclosure may provide to a gene manufacturing system information concerning the first filtered candidate sequence, wherein the gene manufacturing system is operable to use the first filtered candidate sequence to enable a reaction pathway to produce the molecule.

In embodiments, classifying comprises classifying a diversified set of the candidate sequences that satisfy the confidence threshold as the filtered candidate sequences. Classifying the diversified set as the filtered candidate sequences may comprise: clustering, into each cluster of a plurality of clusters, a plurality of candidate sequences that satisfy the confidence threshold; and identifying, as included within the diversified set, at least one candidate sequence from each of at least two clusters of the plurality of clusters. Classifying may further comprise not classifying, as a filtered candidate sequence, a candidate sequence that satisfies the confidence threshold but that is more likely to enable a function different from the desired function. Not classifying may comprises not classifying, as a filtered candidate sequence, a candidate sequence that satisfies the confidence threshold but that is more likely, within a given tolerance, to enable a function different from the desired function.

Embodiments obtain empirical data concerning whether at least one of the filtered candidate sequences enables the desired function, and refine the predictive model using the empirical data. The predictive model may employ machine learning, which may train on the empirical data.

The biological sequences may be enzyme amino acid sequences, and the desired function may be an enzyme-catalyzed reaction. The biological sequences may be enzyme amino acid sequences and the one or more enzymatic functions may be one or more enzyme-catalyzed reactions along one or more reaction pathways, where each reaction pathway produces a molecule. The biological sequences may be nucleotide sequences that code for enzymes, and the desired function may be an enzyme-catalyzed reaction.

The predictive model may be based at least in part upon sequence alignment. The predictive model may be based at least in part upon at least one of the following models: Hidden Markov Model (HMM), artificial neural network, or dynamic Bayesian network.

The molecule may be a bioreachable molecule. The function may be one of a transcription function or a transport function. The molecule may be one of the filtered candidate sequences. One of the filtered candidate sequences may comprise an enzyme amino acid sequence, where the molecule is a bioreachable molecule, and processing comprises catalyzing a reaction using the enzyme amino acid sequence.

The molecule may be a molecule predicted to be a bioreachable molecule, and may be predicted to be a bioreachable molecule by: obtaining a starting metabolite set specifying starting metabolites for the host cell; obtaining a starting reaction set specifying reactions; including in a filtered reaction set one or more reactions from the starting reaction set; and in each processing step of one or more processing steps performed by at least one processor, processing, pursuant to the one or more reactions of the filtered reaction set, data representing the starting metabolites and metabolites generated in previous processing steps, to generate data representing one or more candidate bioreachable molecules.

The host cell may originate from a microbe, a plant, or animal tissue, or may be part of a single-celled organism or a multi-celled organism.

Embodiments of the disclosure may obtain a starting metabolite set specifying starting metabolites for the host cell; obtain a starting reaction set specifying reactions; include, in a filtered reaction set, one or more reactions from the starting reaction set that are indicated as catalyzed by one or more corresponding catalysts; use the system of any one of the preceding embodiments to identify filtered candidate sequences corresponding to one or more of the one or more corresponding catalysts; in each processing step of one or more processing steps performed by at least one processor, process, pursuant to the one or more reactions of the filtered reaction set, data representing the starting metabolites and metabolites generated in previous processing steps, to generate data representing one or more viable target molecules; and provide, as output, data representing the one or more viable target molecules.

### BRIEF DESCRIPTION OF THE DRAWINGS

FIG. 1 illustrates a system for implementing a bioreachable prediction tool according to embodiments of the disclosure.

FIG. 2 is a flow diagram illustrating operation of a bioreachable prediction tool according to embodiments of the disclosure.

FIG. 3 illustrates pseudocode for implementing strict and relaxed enzyme sequence searches according to embodiments of the disclosure.

FIG. 4 illustrates an example of a report that may be generated by the bioreachable prediction tool of embodiments of the disclosure.

FIG. 5 illustrates a hypothetical example of a report of reaction pedigree tracking that may be generated by the bioreachable prediction tool of embodiments of the disclosure.

FIG. 6 illustrates a cloud computing environment according to embodiments of the disclosure.

FIG. 7 illustrates an example of a computer system that may be used to execute instructions stored in a non-transitory computer readable medium (e.g., memory) in accordance with embodiments of the disclosure.

FIG. 8 illustrates an example of a single pathway of the type that may be generated by the biroeachable prediction tool of embodiments of the disclosure. In this example, the molecule tyramine was predicted to be reachable by addition of a single enzymatic step to a host cell. This pathway has been reduced to practice and engineered into host cells to produce tyramine. This pathway's evaluation score is included in the reaction diagram.

FIG. 9 illustrates an example of two distinct pathways of the type that may be generated by the bioreachable prediction tool of embodiments of the disclosure. In this example, both pathways were identified by the bioreachable prediction tool as being able to generate the bioreachable molecule (S)-2,3,4,5-tetrahydrodipicolinate (THDP). The two pathways differ by their use of reducing equivalent types (NADH versus NADPH). One of these pathways has been reduced to practice and engineered into host cells to produce THDP. Each pathway's evaluation score is included in the reaction diagram.

FIG. 10 illustrates an example of a more complex multi-pathway prediction of the type that may be generated by the bioreachable prediction tool of embodiments of the disclosure. Each pathway's evaluation score is included in the reaction diagram.

FIGS. 11A and 11B together illustrates an example of a scoring breakdown that may be generated by the bioreachable prediction tool of embodiments of the disclosure. (FIG. 11B appends to the bottom of FIG. 11A.) In this case, the evaluation data shown was generated during the process of predicting pathways to the molecule (S)-2,3,4,5-tetrahydrodipicolinate (THDP).

FIG. 12 is a flow diagram illustrating the operation of embodiments of the disclosure.

FIGS. 13A-H illustrate an example of identifying at least one sequence to enable tyrosine decarboxylase activity, according to embodiments of the disclosure. FIG. 13A discloses SEQ ID NOS 1-6, respectively, in order of appearance. FIG. 13B discloses SEQ ID NOS 7-10, respectively, in order of appearance.

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

The inventors have recognized that conventional methods for predicting viable target molecules suffer from the following obstacles:

1) Lack of biological parts. This is the single biggest cause of false positive predictions about chemicals that can be biologically generated. Some conventional methods employ existing reaction databases to step through all known metabolic reactions from a feedstock like glucose, and assume all paths can be engineered. However, many reactions do not correspond to a genetic part that can be engineered into a host cell. Typically, reactions are catalyzed by enzymes. Reactions in existing databases may be well-characterized according to their catalyzing enzymes, but many of those enzymes have not had their amino acids sequenced, meaning that there is no established correlation between the enzyme and an associated gene sequence. Without a gene sequence, a host genome cannot be modified to produce the required enzyme. In fact, approximately 25-50% of well-characterized enzymatic reactions have no known associated gene sequence, and thus those enzymes are not useful as biological parts for engineering purposes. The percentage of gene-absent reactions in overall biological databases is likely even higher, since these databases include many reactions that are not well-characterized. The inventors note that, in some cases, catalysts other than enzymes, such as enzyme-nanoparticle conjugates, may be employed. See, e.g., Vertgel A A, et al., Enzyme-nanoparticle conjugates for biomedical applications, Methods Mol. Bio. 2011; 679: 165-82; Johnson P A, et al., Enzyme nanoparticle fabrication: magnetic nanoparticle synthesis and enzyme immobilization, Methods Mol. Biol. 2011; 679: 183-91, all of which are incorporated by reference in their entirety herein. In those cases, parts required to engineer those catalysts into a host cell may or may not be known.

2) Incorrect pathway tracing. Many attempted solutions attempt to trace pathways arbitrarily between molecules. This can lead to a failure to properly track the creation of the target molecule's carbon skeleton. To cite a common example, a path might be traced from glutamine into a reaction that generates a target molecule, and then glutamine would be cited as a part of the path to creating that target molecule. However, in most cases, glutamine is donating a nitrogen group and no carbon, so this tracing is misleading and does not indicate that the target molecule can be made (other errors include tracing connections though other ubiquitous molecules such as ATP or inorganic molecules such as water). These types of pathway tracing errors also lead to an unusably large number of predicted pathways (as if a mapping application allowed all possible street routes through San Francisco, instead of the two to three most direct and useful paths).

3) Assuming bidirectional reactions. Another significant source of errors is failure to account for the thermodynamics/direction of reactions. Thermodynamics dictates that some reactions can only run in one direction. A reaction that only degrades molecule A to molecule B, however, is often predicted by conventional means to run in either direction, so it would be incorrectly predicted that molecule A can be synthesized from B. As a particular example, some bacteria breakdown halogenated compounds such as organochlorides, but cannot run in reverse to create halogenated compounds. Because many biological reactions are dramatically favored to run in only one direction, failure to account for reaction directionality would also create a false positive prediction.

4) Other errors. Not every host can be engineered to produce every target molecule, or be engineered to produce every target molecule with the same set of modifications or likelihood of success, because not all hosts maintain the same set of metabolic pathways.

Embodiments of the disclosure overcome the limitations of conventional methods. Embodiments of the disclosure may provide, in a target-agnostic fashion, every chemical that likely can be biologically generated given a set of starting constraints (e.g. particular host cell, number of reaction steps, whether only reactions with gene-sequenced enzymes allowed). This creates a “bioreachable list,” a list of viable target chemicals. These target chemicals and their associated structures can be provided to professional chemists, who can review the chemical utility of the molecules without having to consider the biology required to create them. After particular target molecules are selected, their formulas and reaction pathways may be provided to a gene manufacturing system to modify the gene sequence of the host cell to produce the selected target molecules.

The bioreachable prediction tool of embodiments of the disclosure obtains a starting metabolite set specifying starting metabolites for the host cell. In embodiments, the starting metabolite set specifies core metabolites, the core metabolites including metabolites indicated by at least one database as produced by an un-engineered host under specified conditions. In embodiments, the host has not been subjected to genetic modification.

In embodiments, the bioreachable prediction tool obtains a starting reaction set specifying reactions. In embodiments, the tool includes in a filtered reaction set one or more reactions from the starting reaction set that are indicated in at least one database as catalyzed by one or more corresponding catalysts, e.g., enzymes, that are themselves indicated as likely available to catalyze the one or more reactions that may take place in the host cell.

A catalyst is likely “available to catalyze” a reaction in a host cell if the bioreachable prediction tool determines information from, e.g., public or proprietary databases, indicating that the catalyst may be introduced into the host either by engineering the catalyst into the host (e.g., by modifying the host genome, adding a plasmid) or via uptake of the catalyst from the growth medium in which the host is grown.

More specifically, this disclosure refers to a part, such as a catalyst, as being “engineered” into a host cell when the genome of the host cell is modified (e.g., via insertion, deletion, replacement of genes, including insertion of a plasmid coded for production of the part) so that the host cell produces the catalyst (e.g., an enzyme protein). If, however, the part itself comprises genetic material (e.g. a nucleic acid sequence acting as an enzyme), the “engineering” of that part into the host cell refers to modifying the host genome to embody that part itself.

A part is likely “available to be engineered” into the host cell if the bioreachable prediction tool determines information indicating that the part can be engineered in the host. For example, according to embodiments, the tool would determine information indicating that an enzyme is likely available to be engineered into a host if the enzyme is found to be engineerable into the host, e.g., as indicated by annotation in a public or proprietary database accessed by the BPT tool. If there is evidence that at least one amino acid sequence is known (e.g., found in one of the above databases) to catalyze the reaction (in any host), then skilled artisans would be able to derive the corresponding genetic sequence used to code the amino acid sequence, and modify the host genome accordingly. If the likely available part is an enzyme, the tool can select a set of enzyme sequences predicted as highly likely to catalyze a reaction needed to make the molecule, where an enzyme sequence may be represented as a protein amino acid sequence or genetically as as DNA or RNA, and may be native or heterologous. In this context and in the claims, “likely” means more probable than not, i.e., having a greater than 50% likelihood.

In each processing step of one or more processing steps leading to prediction of a bioreachable molecule, the bioreachable prediction tool processes, pursuant to the one or more reactions of the filtered reaction set, data representing the starting metabolites and metabolites generated in previous processing steps, to generate data representing one or more viable target molecules. The tool provides, as output, data representing the one or more viable target molecules.

In embodiments, the bioreachable prediction tool determines a degree of confidence as to whether a corresponding catalyst is available to catalyze the one or more reactions in the host cell, e.g., available to be engineered into the host cell to catalyze the one or more reactions. The degree of confidence may include, for example, at least a first degree of confidence or a second degree of confidence higher than the first degree of confidence. The tool may include, in the filtered reaction set, one or more reactions from the starting reaction set that are indicated in at least one database as catalyzed by one or more corresponding catalysts that are themselves determined to be available, with the second degree of confidence, to catalyze the one or more reactions in the host cell, e.g., determined to be available, with the second degree of confidence, for engineering into the host cell to catalyze the one or more reactions.

In embodiments of the disclosure, the bioreachable prediction tool generates an indication of the difficulty of producing one or more of the viable target molecules. The indication of difficulty may be based upon thermodynamic properties, reaction pathway length for the one or more viable target molecules, or a degree of confidence as to whether a catalyst is available to catalyze one or more corresponding reactions along one or more first reaction pathways to one or more of the viable target molecules.

In embodiments of the disclosure, after generating data representing one or more viable target molecules in a particular processing step and before the next processing step, the bioreachable prediction tool removes from the filtered reaction set any reactions associated with generating the data representing one or more viable target molecules in the particular processing step.

In embodiments, the tool generates a record of one or more reaction pathways (i.e., pedigrees) leading to each viable target molecule. In embodiments, generating a record comprises not including in the record reaction pathways from ubiquitous metabolites. In embodiments, the tool generates a record of the step in which data representing a viable target molecule is generated. In embodiments, the tool generates a record of the shortest reaction pathway from the starting metabolite set to each viable target molecule.

Instead of determining viable target molecules given a single host cell, it may be desired to identify one or more host cells in which to produce a given viable target molecule. For example, a customer may ask the user of the tool to determine the optimum host cell within a plurality of hosts in which to produce the target molecule. In embodiments, the bioreachable prediction tool is run for a plurality of host cells, and generates data representing one or more viable target molecules (bioreachable candidate molecules), according to any of the methods described herein, for each host cell of the plurality of host cells. In such embodiments, for a given viable target molecule, the tool determines at least one of the plurality of host cells that satisfies at least one criterion, such as a given predicted yield of the viable target molecule produced by a given host cell or a given number of processing steps predicted as necessary to produce the given viable target molecule in a given host cell. The tool provides, as output, data representing the host cells determined to satisfy the at least one criterion.

As described for embodiments above, the tool may generate a record, including, e.g, thermodynamic properties, of one or more reaction pathways (i.e., pedigrees) leading to each target molecule produced by each host cell. Based on the above embodiments running the tool for the plurality of host cells, the tool may store associations between host cells, target molecules, and pedigrees in a database as a library, which may include annotations specifying parameters such as yield, number of processing steps, availability of catalysts to catalyze reactions in the reaction pathways, etc.

In embodiments, if the tool has access to such a library, the tool need not be run to identify multiple host cells in which to produce a given viable target molecule. Instead, in such embodiments, the tool may use the pedigrees from the library, which may include annotation data concerning associations among the hosts, target molecules, and reactions. The tool may identify at least one target host cell from among the one or more host cells based at least in part upon evidence, from, e.g., public or proprietary databases or from the library, that all the catalysts predicted to catalyze reactions in at least one reaction pathway leading to production of the target molecule in the at least one target host cell are likely available to catalyze all such reactions. In embodiments, the tool may determine target hosts based upon the target hosts requiring less than a threshold number of reaction steps within the reaction pathways that are predicted as necessary to produce the target molecule.

Some reaction enzymes may not have a known associated amino acid sequence or genetic sequence (“orphan enzymes”). In such cases, the tool may instead bioprospect the orphan enzymes to predict their amino acid sequences, and, ultimately, their genetic sequences, so that the newly-sequenced enzymes may be engineered into the host cell to catalyze one or more reactions. The tool may include the reactions corresponding to the newly-sequenced enzymes as members of the filtered reaction data used for bioreachable molecule finding.

In embodiments, the bioreachable prediction tool provides to a “factory,” e.g, a gene manufacturing system, an indication of one or more genetic sequences associated with one or more reactions in a reaction pathway leading to a viable target molecule. In embodiments, the gene manufacturing system embodies the indicated genetic sequences into the genome of the host, to thereby produce an engineered genome for manufacture of the target molecule. In embodiments, the tool provides to the factory an indication of one or more catalysts for the factory to introduce the one or more catalysts into the growth medium of the host cell for production of the target molecule.

In embodiments, the bioreachable prediction tool includes, in the filtered reaction set, reactions from the starting reaction set based at least in part upon whether the one or more reactions are spontaneous, based at least in part upon their directionality, based at least in part upon whether the one or more reactions are transport reactions, or based at least in part upon whether the one or more reactions generate a halogen compound.

In embodiments of the disclosure, the bioreachable prediction tool obtains a starting metabolite set specifying starting metabolites for the host cell, and obtains a starting reaction set specifying reactions specific to the host. In embodiments of the disclosure, the bioreachable prediction tool includes in a filtered reaction set one or more reactions that are indicated as spontaneous in at least one database. In each processing step of one or more processing steps, the tool processes, pursuant to the one or more reactions of the filtered reaction set, data representing the starting metabolites and any metabolites generated in previous processing steps, to generate data representing one or more viable target molecules in each step. In embodiments, the tool provides, as output, data representing the one or more viable target molecules.

### System Design

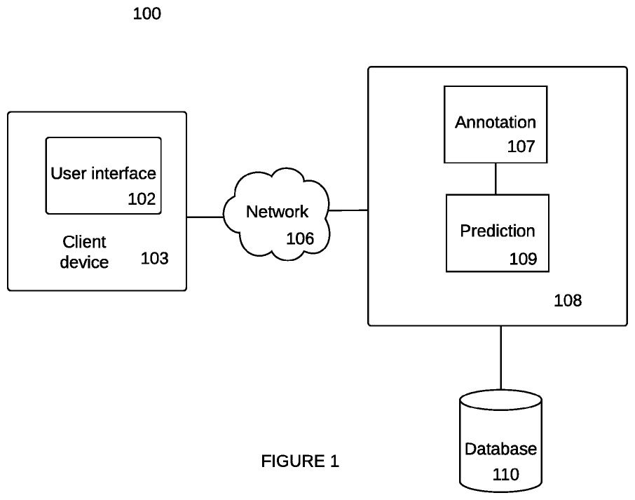

FIG. 1 illustrates a distributed system 100 of embodiments of the disclosure. A user interface 102 includes a client-side interface such as a text editor or a graphical user interface (GUI). The user interface 102 may reside at a client-side computing device 103, such as a laptop or desktop computer. The client-side computing device 103 is coupled to one or more servers 108 through a network 106, such as the Internet.

The server(s) 108 are coupled locally or remotely to one or more databases 110, which may include one or more corpora of molecule, reaction, and sequence data. The reaction data may represent the set of all known metabolic reactions. In embodiments, the reaction data is universal, i.e., not host-specific.

The molecule data includes data on metabolites—reactants involved in the reactions contained in the reaction data as either substrates or products. In embodiments, the data on metabolites includes data on host-specific metabolites, such as core metabolites, known in the art to be produced in particular host cells. In some embodiments, some core metabolites were determined to be produced by a particular host through empirical evidence gathered by the inventors. These host-specific metabolite sets were identified through various methods such as metabolomics analysis of the host cell or by identifying enzyme-coding genes that are essential under certain growth conditions, and inferring the presence of metabolites produced by the enzymes coded by those genes. The molecule data may be tagged with annotations representing many features, such as host cell, growth medium characteristics, and whether a molecule is a core metabolite, a precursor, ubiquitous, or inorganic.

The database(s) 110, e.g., UniProt, may also include data on whether a catalyst may be introduced into a host cell via uptake of the catalyst from a growth medium in which the host is grown.

The sequence data may include data for the reaction annotation engine 107 to annotate reactions in the reaction data set as to whether a reaction is likely known to correspond to sequences, e.g., enzyme or genetic sequences, for engineering the reaction into a host cell. For example, the sequence data may include data for annotating reactions in the reaction data as to whether a reaction is catalyzed by an enzyme for which the corresponding amino acid sequence is likely known. If so, then, through methods known in the art, a genetic sequence for coding the enzyme can be determined. In embodiments, for purposes of determining viable target molecules, the reaction annotation engine 107 does not need to know the sequence data itself, but rather only whether a sequence is likely known to exist for the catalyst. The reaction annotation engine 107, described below, may compile the sequence data from databases such as UniProt, which include sequence data for enzymes that catalyze reactions indicated as having associated coding sequences. The sequence data may also be used during the enzyme selection step to both train models and provide a source of possible predicted sequences.

In embodiments, the server(s) 108 includes a reaction annotation engine 107 and a bioreachable prediction engine 109, which engines together or separately form the bioreachable prediction tool of embodiments of the disclosure. Alternatively, the software and associated hardware for the annotation engine 107, the prediction engine 109, or both may reside locally at the client 103 instead of at the server(s) 108, or be distributed between both client 103 and server(s) 108. The database(s) 110 may include public databases such as UniProt, PDB, Brenda, BKMR, and MNXref, as well as custom databases generated by the user or others, e.g., databases including molecules and reactions generated via synthetic biology experiments performed by the user or third-party contributors. The database(s) 110 may be local or remote with respect to the client 103 or distributed both locally and remotely. In some embodiments, the annotation engine 107 may run as a cloud-based service, and the prediction engine 109 may run locally on the client device 103. In embodiments, data for use by any locally resident engines may be stored in memory on the client device 103.

### System Operation

Obtaining the starting metabolite lists and starting reaction data sets

Inputs to the bioreachable prediction process include information such as starting metabolite list, starting reaction list, host cell, and baseline conditions, such as fuel level for the host (e.g., minimal or rich growth medium) and environmental conditions such as temperature. The annotation engine 107 may assemble metabolite and reaction data along with associated annotations from the database(s) 110.

Through the user interface 102, a user may specify the database(s) 110 from which to obtain information for the starting metabolite and reaction lists. For example, reactions and host-specific metabolites may be obtained from public databases such as KEGG, Uniprot, BKMR, and MNXref. (Those skilled in the art will recognize from the context of the discussion that references in this specification and the claims to “metabolites,” “reactions,” and the like may in many instances in fact refer to data representing those physical objects or processes and not the physical objects or processes themselves.)

### Starting Metabolite List

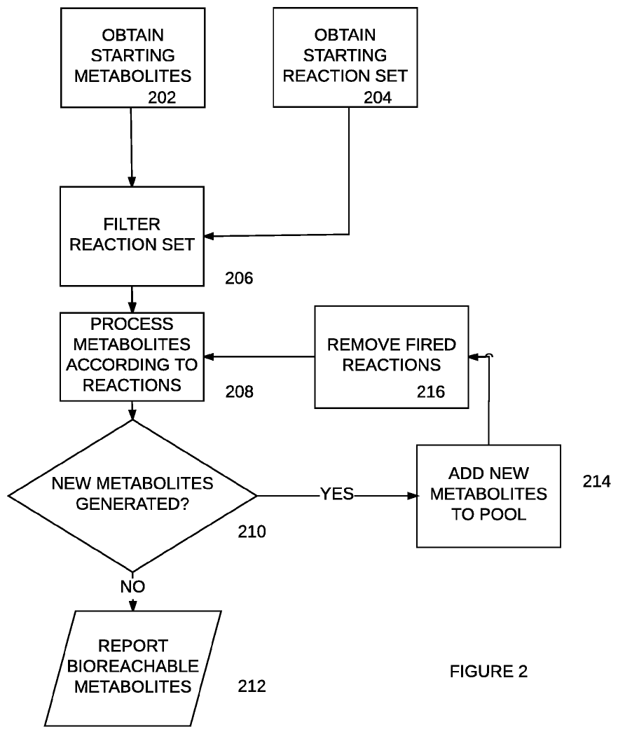

Referring to FIG. 2, in embodiments the reaction annotation engine 107 obtains or itself aggregates from the database(s) 110 a host-specific starting metabolite file comprising a list of chemical compounds (starting, intermediate, and final products) that are expected to be present during the growth of the host cell at a particular time or during a particular time interval under given growth conditions (202). The default growth condition may be a minimal growth medium, because this is the most conservative approach for selecting the starting metabolites. In embodiments, the reaction annotation engine 107 may provide the metabolite file as a starting metabolite list to the prediction engine 109.

In embodiments, the reaction annotation engine 107 may determine or template (off of similar microbes) the starting metabolites based on growth data for the host cell or for a similar cell. This approach is similar to approaches used to annotate the genomes of microbes in systems such as the RAST system, or to predict metabolic pathways in the BioCyc database collection. This approach uses the genome annotation for a given host cell to make a best guess at which metabolic pathways are present, and then assumes the presence of all the constituent reactions, and their metabolites, in those pathways. In the case of BioCyc databases, the existing genome annotation is used to identify the putative presence of individual enzymes (and thus their reactions). A rule-based system is then used to infer the presence of entire metabolic pathways based on the presence of (some of) their substituent reactions.

Having a starting metabolite list that is specific to the host cell is a distinguishing starting point for embodiments of the disclosure. Whereas other conventional approaches make generic predictions about targets that can be made, this customizable step of embodiments of the disclosure avoids the problem of making incorrect predictions about which target molecules can be made (or how they can be made) due to differences in the biology of host cells.

In embodiments, the user may instruct the reaction annotation engine 107 to retrieve the starting metabolites from existing databases or datasets, such as MNXref, KEGG or BKMR, based upon querying the databases or datasets with parameters such as host cell and growth medium, and, in some embodiments, via cross-indexing those databases with relevant model cell databases or other indications of the presence of specific metabolites. So far, for particular industrial hosts the assignees have created typical starting metabolite files on the order of 200-300 metabolites. As noted above, data objects representing metabolites in the public databases and the lists formed by the annotation engine 107 may include annotations including metadata such as host cell, growth medium type, and whether the metabolite is a core metabolite, a precursor, inorganic, or ubiquitous.

Core metabolites are the starting (e.g., substrate), intermediate and final metabolites natively found in a genetically-unmodified cells for given baseline conditions, such as the richness of the growth medium. Each core metabolite (e.g., amino acid) in the biomass of a microorganism like E. coli may be generated in the cell's core metabolism from one of eleven precursor metabolites, and may be fundamentally generated from whatever carbon input is provided to the genetically-unmodified cell. In embodiments, the user may select a starting metabolite set of select core compounds tagged with their precursor dependencies from databases such as MNXref, KEGG, ChEBI, Reactome, or others.

As their name suggests, inorganic metabolites, such as ammonium, do not include carbon, and thus cannot contribute a carbon atom to new products of the metabolism. Accordingly, the reaction annotation engine 107 may exclude inorganic metabolites from the starting metabolite set.

Some metabolites are ubiquitous, i.e., they are found in many reactions. They include molecules like ATP and NADP. Typically, ubiquitous molecules do not contribute carbon to the target product, and thus would not be part of any metabolic pathway to the target. Accordingly, the reaction annotation engine 107 may exclude ubiquitous metabolites from the starting metabolite set. Ubiquitous molecules can be manually designated in annotations based on expert evaluation or identified by determining what molecules participate in reactions beyond a particular threshold number. One heuristic flags all molecules that appear in the reaction set at numbers greater than the size of a typical core metabolite input (e.g., 300). For example, in one data set ATP appears in 2,415 of approximately 31,000 reactions, NADH appears in 2,000 reactions, and NADPH appears in 3,107 reactions, which places them above the core metabolite count and earns them all the “ubiquitous” tag.

### Starting Reaction Data Set

The reaction annotation engine 107 obtains a starting reaction data set as the basis for prediction of viable target molecules (204). The user may specify how to build the starting reaction data set, or the user may instruct the annotation engine 107 to obtain the data directly from a public database 110 or a proprietary database 110, such as a custom database previously created by the user or others. In one embodiment, the annotation engine 107 may import the full reaction set (approximately 30,000 reactions) from the MetaNetx reaction namespace (MNX) of MNXref. In other embodiments, the annotation engine 107 may import and merge the reaction sets (approximately 22,000 total reactions) from MetaCyc and KEGG, or other public or private databases.

In embodiments, the reaction annotation engine 107 may build the starting reaction data set by selectively aggregating the information obtained from the database(s) 110. For example, BKMR provides information whether a reaction is spontaneous. The annotation engine 107 may use known mappings to map BKMR reaction IDs to IDs in MNXref for corresponding reactions. In other examples, KEGG or MetaCyc and their IDs may be employed instead of BKMR and its IDs. Using this association, the reaction annotation engine 107 may then create a custom reaction list in database(s) 110 using the existing annotations from MNXref (e.g., core, ubiquitous), along with a corresponding spontaneous reaction tag from BKMR. Similarly, through mapping corresponding IDs, the annotation engine 107 may associate reactions in MNXref with annotations in UniProt to obtain tags for whether a reaction is a transport reaction or whether a reaction substrate or product contains a halogen, and incorporate those tags into the annotations for the reaction in the custom reaction list in database(s) 110. (Identifying halogenated compounds is a heuristic for identifying reactions that run in the wrong direction, since most halogen-related reactions concern breaking down a chemical.)

Along these lines, the reaction annotation engine 107 may use associated IDs across databases to aggregate data from the databases to build a database 110 storing starting reaction sets with custom annotations, such as whether the reaction is spontaneous, runs in only one direction due to thermodynamics, contains a halogen (related to determining directionality), contains a ubiquitous metabolite, is a transport reaction, is unbalanced (that is, the two sides of the chemical reaction do not maintain elemental balance, suggesting the reaction is improperly written in the source database and should be ignored), is incompletely characterized in available databases, is associated with enzymes tagged with an indicator that the enzyme is associated with a known amino acid sequence or genetic sequence coding the enzyme, or is catalyzed by source enzymes likely to have transmembrane domains, among other tags. Through the annotation engine 107, the user may thus assign annotations to all of the approximately 30,000 reactions in the MNXref database, for example. As described below, the user may then configure criteria to filter this master file into individual lists for each annotation feature or any combination thereof.

### Bioreachable Molecule Prediction

Referring to the flow diagram of FIG. 2, the following describes examples of the operation of the prediction engine 109 of embodiments of the disclosure. The prediction engine 109 predicts which chemicals can be created via, e.g., genetic engineering, in an arbitrarily selected host cell. The prediction engine 109 may take as inputs a starting metabolite file, a starting reaction data set, and a sequence database. The sequence database may store the amino acid sequences for catalytic compounds (such as enzymes), or the genetic sequences that encode catalytic compounds. Embodiments of the disclosure use the sequence database to determine the presence or absence of an amino acid sequence or genetic sequence for each reaction. In such embodiments, the sequence database need not include the sequences themselves, as long as the catalysts are tagged as having an enzyme or genetic part available or not. Along with a list of bioreachable candidate molecules, the prediction engine 109 produces for a specified host cell “pedigrees” (reaction pathways) of the reactions leading to production of each molecule from the starting metabolites, e.g., the host's core metabolites in some embodiments.

In particular, the predictions can be tuned based on a number of parameters, such as likely availability of catalysts to catalyze reactions, (e.g., likely availability of genetic parts to be engineered into the host cell or likely availablilty of catalysts to be introduced into the host cell via uptake from a growth medium in which the host cell is grown), maximum number of reaction steps allowed (starting from the starting metabolites), types of parts or chemical reactions to be allowed, and other selectable features. The prediction engine 109 also helps predict the approach to, and difficulty in designing target molecules by predicting the potential paths from core metabolites to each target molecule.

### Filtered Reaction Data Set

In embodiments, the prediction engine 109 creates a filtered and validated reaction data set (RDS). Using the reactions characterized by the reaction annotation engine 107, the prediction engine 109 may filter the reactions to a desired level of validation, e.g., level of confidence that a coding sequence for the reaction enzyme exists (206). This is a step in fine tuning the accuracy of the predictions, and for controlling the primary source of false positive predictions. In an example mentioned above, the inventors generated the RDS for one bioreachable list by importing and annotating the full reaction set (approximately 30,000 reactions) from the MetaNetx reaction namespace (MNX) of MNXref. A similar approach could be applied to other publicly available reaction databases such as KEGG, Reactome, and MetaCyc.

Based upon the inventors' experience, 25-50% of the reactions in the most popular public databases may not have any known associated biological parts. For example, the amino acid sequences of enzymes for catalyzing the reactions, or their accompanying genetic sequences, may be unknown. Without the enzyme sequence information, a bioreactor would not be able to perform the reactions employing those enzymes, thus rendering the reaction information useless for engineering purposes. Even if only one enzyme within a pathway lacks a known gene sequence, then the entire pathway cannot be engineered into a host.

To deal with this deficiency, the prediction engine 109 may filter the reactions through a series of validation tests using publicly available or custom enzyme data. One public database is UniProt, which is large, open access, and reliably curated. Others include the RCSB Protein Data Bank (PDB) and GenBank. In some public databases such as MNXref, UniProt, Brenda, or PDB, reactions may be tagged with an Enzyme Commission (EC) number, which is a numerical classification for enzymes based on the reactions they catalyze. Some databases, such as UniProt or PDB, store EC number tags only for reactions for which the gene sequence coding the catalyzing enzymes are known. Other databases, such as KEGG and MetaCyc, include EC numbers for enzymes for which the gene sequence is not known.

Thus, depending upon the database, an EC number may or may not indicate the existence of a known enzyme gene sequence. Approximately, 20-25% of reactions with EC numbers have no associated enzyme coding sequence. In some cases, EC numbers are used to annotate multiple specific chemical transformations (there is a one-to-many relationship between EC numbers and chemical reactions), so that the presence of an enzyme sequence associated with an EC number does not mean that every reaction associated with that EC has a valid associated sequence. Thus, the presence of an EC tag on an enzyme activity is not a reliable general indicator of the presence of a gene sequence for that enzyme, but it can be applied to certain databases to determine if a sequence is reasonably likely to be present for that enzyme. Some databases also have separate fields (e.g. the “catalytic activity” field in UniProt) that explicitly describe particular chemical reactions as known to be definitively catalyzed by a given amino acid sequence (thus having a known genetic sequence for coding the enzyme catalyst). Such reactions are referred to herein as annotated as “definitively sequenced.”

The prediction engine 109 may determine a degree of confidence as to whether a catalyst is available to catalyze a reaction in the host cell (e.g., available to be engineered into the host cell to catalyze the reaction). For example, based on the differences in certainty that enzyme coding sequences are known, the prediction engine 109 may execute, in some embodiments, a “strict” search or a “relaxed” search for enzyme coding sequences against annotations in the reaction data set. For a strict search, the prediction engine 109 may select, for example, only reactions annotated as being definitively sequenced.

In embodiments, the prediction engine 109 may factor, into the degree of confidence as to whether a catalyst is available to catalyze a reaction, the degree of confidence (e.g., expect-value) that a sequence (e.g., enzyme amino acid sequence, nucleotide sequence) enables a desired function in a host cell, as described in embodiments below.

For a relaxed search, the prediction engine 109 may select, for example, reactions annotated as having an EC number that is associated with known enzyme coding sequences or (Boolean non-exclusive OR) reactions that are annotated as “definitively sequenced” in the sequence database, from annotations derived from databases such as MetaCyc. The prediction engine 109 records whether any gene or amino acid sequences are found for the reactions, for either level of confidence. For example, the prediction engine 109 may annotate the reaction with a tag indicating that it satisfies the relaxed search, but not the strict search.

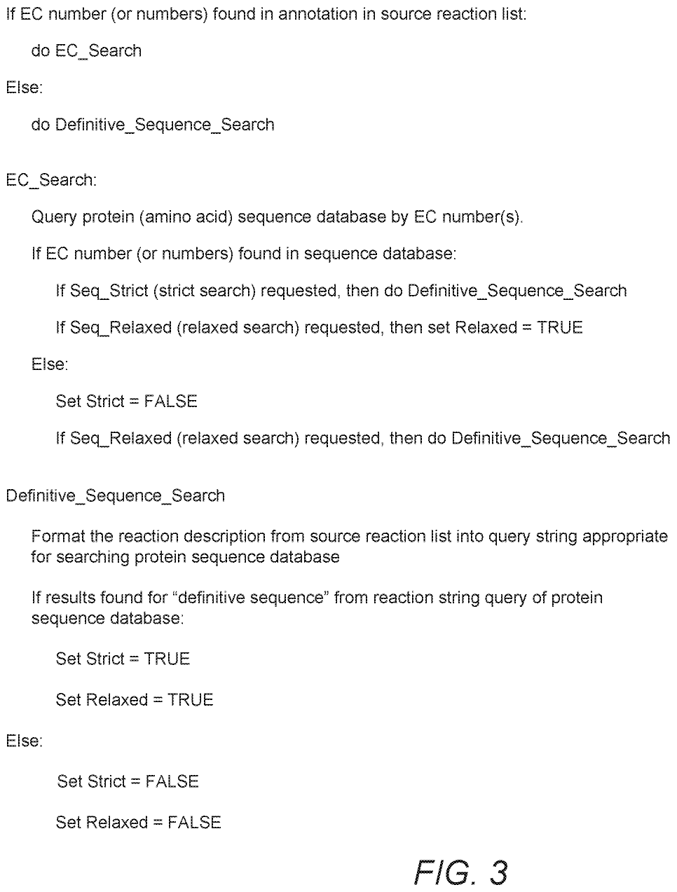

FIG. 3 illustrates exemplary pseudocode for implementing strict and relaxed enzyme sequence searches against databases, such as MNXref and UniProt, according to embodiments of the disclosure. The pseudocode describes the logic used by a heuristic for determining whether a sequence exists for an enzyme. This embodiment provides four levels of confidence. The code shows first determining whether the reaction data set annotations include at least one EC number. If so, then the code calls for searching the sequence database for EC numbers. If a strict search is being conducted, then the code calls for searching the sequence database for reactions that are definitively sequenced. If a relaxed search is being conducted, then the code sets the Relaxed annotation tag for the reactions having associated EC numbers to TRUE.

If the initial step determines that the reaction data set annotations (a) do not include an EC number or (b) (as mentioned above) the EC sequence search finds an EC number in the sequence database and a strict search is being conducted, then the code calls for searching the sequence database for reactions that are definitively sequenced. If that search finds a reaction as definitively sequenced, then the code sets both the Strict and Relaxed annotations for that reaction as TRUE. If not, then the code sets both those annotations for that reaction as FALSE.

In sum, the output of this heuristic are two annotation tags for each reaction: Strict and Relaxed. This heuristic provides four levels of confidence, as described below:

Strict=TRUE→very high confidence a sequence exists
Strict=FALSE→moderate confidence a sequence does not exist (expect some false negatives)
Relaxed=TRUE→moderate confidence a sequence exists (expect some false postivies)
Relaxed=FALSE→very high confidence a sequence does not exist

The inventors have found that running a relaxed search results in less than a 20% false positive rate, whereas running a strict search against the catalytic activity field in UniProt results in a significant false negative rate. Thus, it may be better to err slightly on the side of a relaxed search. The “relaxed” and “strict” tags are just two potential methods of handling sequence-based filtering. The bioreachable prediction tool is amenable to any sequence-based tagging (and thus filtering) approach, including more permissive methods such as identifying the presence of sequences with appropriate motifs for the target activity or more stringent methods such as requiring the presence of a directly-literature-supported activity-sequence link in a heavily curated database such as MetaCyc.

As an alternative or in addition to the sequence-based filtering, the prediction engine 109 may filter (i.e., select or not select) reactions based upon any combination of the annotations discussed above with respect to the annotation engine 107, such as reaction directionality, or whether a reaction is a spontaneous reaction, a transport reaction, or contains a halogen. The prediction engine 109 may perform filtering based on user configuration through the user interface 102 or default settings. In embodiments, the prediction engine 109 may apply different filters in different reaction steps along the simulated metabolic pathways. As an example of default settings, they may be: reaction has a sequence based on relaxed criteria; exclude all transport reactions; only include reactions containing halogens if the reactions have a sequence; include all spontaneous reactions regardless of the above attributes.

If a reaction is spontaneous, the reaction will occur automatically without the need to engineer the host genome to produce an enzyme to catalyze the spontaneous reaction. Since the reaction is known to occur under given conditions for a given host, the prediction engine 109 can predict that the spontaneous reaction products will be produced.

As noted above, inorganic molecules do not contribute carbon and ubiquitous molecules are unlikely to contribute carbon to target metabolites. Thus, eliminating ubiquitous and inorganic molecules from those used as starting metabolites heuristically provides a high confidence level that the prediction engine 109 will follow valid metabolic pathways in predicting viable target molecules. Accordingly, the prediction engine 109 does not treat ubiquitous or inorganic molecules as limited in a reaction. That is, they are assumed to always be available to the reactions in which they participate.

### Metabolite Prediction

Referring to FIG. 2, the prediction engine 109 may perform a stepwise simulation to predict which metabolites would be formed, given a substrate of input metabolites processed according to the reactions in the filtered RDS (208). (A chemical reaction operates on an input “substrate” (e.g., set of molecules) to produce chemical products.) The operation of the prediction engine 109 of embodiments of the disclosure may be described as follows:

Step 0: Initially, only core metabolites are present in the simulated host cell. They form the current substrate for the reactions in the next step.

Step 1: The prediction engine 109 determines whether the core metabolites from step 0 match one side of any of the chemical equations within the filtered reaction set (RDS), and whether a reaction can take place in a given direction (based on directional/thermodynamic annotation), to thereby determine which reactions would fire to produce chemicals on the other side of the reaction equation (208). The prediction engine 109 determines whether any new metabolites are produced by the fired reactions (210).

If the prediction engine 109 determines that no new metabolites have been predicted (210), then the prediction engine 109 ends the prediction process, and reports the results (212).

Conversely, if the prediction engine 109 determines that new metabolites would be formed (210), the prediction engine 109 adds the new metabolites to the substrate pool (214). The updated substrate pool now includes the core metabolites and the newly predicted metabolites from step 1.

The prediction engine 109 records the metabolites and fired reactions in each step, and also removes the fired reactions from the filtered RDS (step 216). This removal prevents the same reactions from being fired in subsequent steps, to thereby avoid a reaction and its resulting metabolite(s) from being identified as present in a subsequent step. Each reaction is simulated only once throughout all steps of the process. This comports with engineering best practices that generally focus on the shortest path (fewest number of steps) to reach a metabolite—longer pathways to the same metabolite are typically suboptimal. Along with the metabolites and reactions within each step, the prediction engine 109 records the step in which a metabolite is made (i.e., predicted to be made). That step represents the metabolic path length to generating the metabolite. Note that a metabolite may appear as a product in multiple steps if it is created via distinct reactions. This fact allows the prediction engine to identify usefully distinct pathways, where the same metabolite is reached by distinct reactions.

Step 2: The prediction engine 109 then returns to step 208 using the now updated substrate pool of metabolites as inputs to run against the filtered RDS (with fired reactions now removed) to predict whether any reactions would fire to produce new metabolites.

After multiple iterations, the pool of metabolites grows whereas the pool of available reactions shrinks. Eventually, the process may run to saturation because no more metabolites are left that can fire a reaction that remains in the filtered RDS. In experiments by the inventors, approximately 10,000 filtered reactions may result in several thousand metabolites after all iterations. Alternatively, the prediction engine 109 may be configured to specify the number of allowed reaction steps before halting the predictions and reporting the results (212). The limitation on number of reaction steps reflects real-world engineering, which would typically limit the number of cycles.

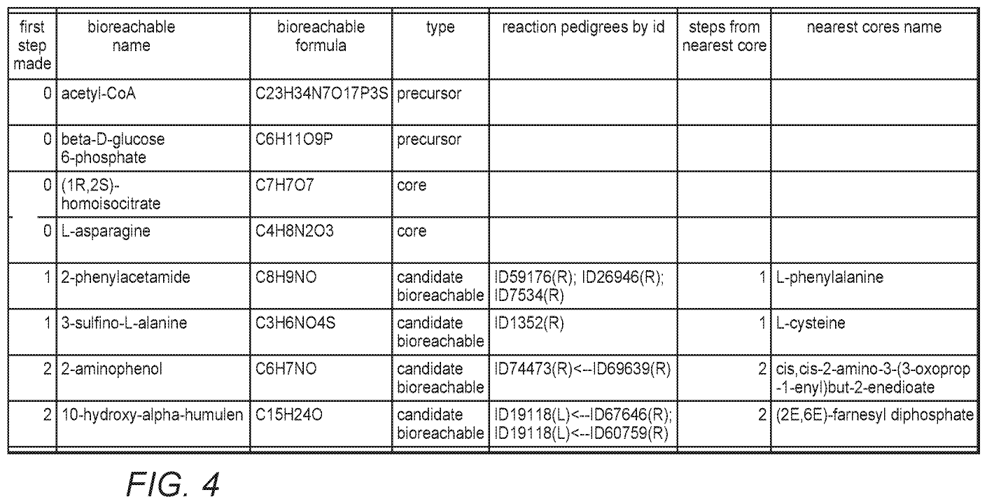

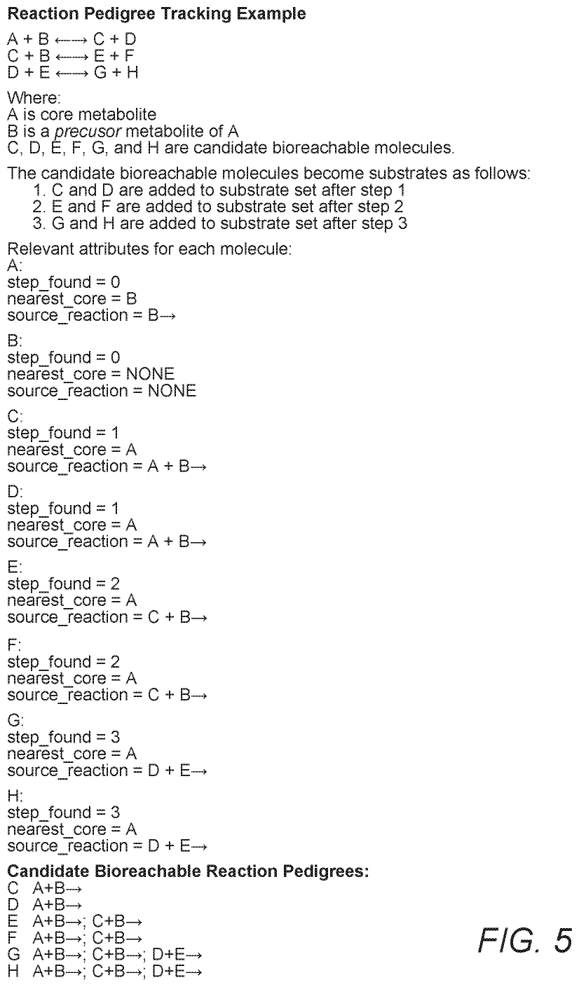

FIGS. 4 and 5 illustrate examples of reports that may be generated by embodiments of the disclosure. FIG. 4 shows, for each processing step, the metabolites generated (bioreachable name), their chemical formulas, the type of metabolite (e.g., core, precursor, candidate bioreachable produced by a reaction), the reaction pedigrees of the metabolites as denoted by a unique reaction ID such as an ID used in well-known databases (which also shows whether the left (“L”) or right (“R”) side of the reaction fired), the number of reaction steps needed from the nearest core metabolite to produce the candidate bioreachable molecule, and the name of the nearest core metabolite for each candidate bioreachable molecule. Note that the only molecules in step 0 are from the starting metabolite list (e.g., cores, precursors).

FIG. 5 illustrates a hypothetical example of reaction pedigree tracking. Stepwise the reactions are as follows:

Step 1: A+B←→C+D

Step 2: C+B←→E+F

Step 3: D+E←→G+H

The attributes in this example include: whether the metabolite generated in the step is a core; the step in which the metabolite is found; the nearest core metabolite to the generated metabolite, as measured by distance in number of steps; and the reaction pedigree denoting the chemical reaction fired to produce the metabolite. Metabolite A is a core metabolite and B is a precursor metabolite present in the biomass of the host at Step 0. Thus they have no reaction pedigree.

C and D are shown as produced in Step 1 by the reaction A+B in the reaction pedigree (source_reaction). The nearest core to both C and D is A. C and D are added to the substrate along with cores A and B.

E and F are shown as produced in Step 2 by the reaction C+B. The nearest core to both E and F is A. E and F are added to the substrate along with cores A and B and bioreachable products C and D.

G and H are shown as produced in Step 3 by the reaction D+E. The nearest core to both G and H is A.

Embodiments of the disclosure may also output the pathway (also known as the “pedigree” sequence of reactions) for each metabolite as follows:

C: A+B→

D: A+B→

E: A+B→; C+B→

F: A+B→; C+B→

G: A+B→; C+B→; D+E→

H: A+B→; C+B→; D+E→

Pathway filtering. In embodiments, given a host cell, a target molecule, and the reaction pedigrees of the pathways leading to the given target molecule, the prediction engine 109 may selectively filter the pathways to identify pathways based on given parameters, such as path length (e.g., number of reaction processing steps from starting metabolite to target molecule). The prediction engine 109 may provide, as output, data representing the identified reaction pathways.

Host cell selection. Instead of determining viable target molecules given a single host cell, it may be desired to identify one or more host cells in which to produce a given viable target molecule. In embodiments, the prediction engine 109 generates data representing viable target molecules, according to methods described above, for not just one host cell, but for a plurality of host cells. In such embodiments, for a given viable target molecule, the prediction engine 109 determines at least one of the plurality of host cells that satisfies at least one criterion. For example, using the reaction pedigree data, the prediction engine 109 may select a host cell based upon the number of processing steps predicted as necessary to produce the given viable target molecule in that host cell. As another example, the prediction engine 109 may select a host cell based upon the predicted yield of the viable target molecule produced by that host cell. Predicted yield may be derived in a number of ways, including Flux-Balance Analysis (FBA) based on a separate model for each potential host, simple elemental yield modeling, and precursor-based percent yield estimates. The prediction engine 109 provides, as output, data representing the host cells determined to satisfy the at least one criterion.

As described for embodiments above, the prediction engine 109 may generate a record of one or more reaction pathways (i.e., pedigrees) leading to each target molecule produced by each host cell. Based on the above embodiments that run the tool for the plurality of host cells, the reaction annotation engine 107 may store associations between host cells, target molecules, and pedigrees in a database as a library, which may include annotations specifying parameters such as yield, number of processing steps, availability of catalysts to catalyze reactions in the reaction pathways, etc. Alternatively, the library may be obtained from a third party.

In embodiments, if the prediction engine 109 has access to such a library, the tool need not be run to identify multiple host cells in which to produce a given viable target molecule. Instead, in such embodiments, the prediction engine 109 may use the pedigrees from the library, which may include annotation data concerning associations among the hosts, target molecules, and reactions. The prediction engine 109 may identify at least one target host cell from among the one or more host cells based at least in part upon evidence, from, e.g., the library or public or proprietary databases, that all the catalysts predicted to catalyze reactions in at least one reaction pathway leading to production of the target molecule in the at least one target host cell are likely available to catalyze all such reactions in the at least one reaction pathway. In embodiments, the prediction engine 109 may determine target hosts based upon the target hosts requiring less than a threshold number of reaction steps within the reaction pathways that are predicted as necessary to produce the target molecule.

Bioprospecting. Some reaction enzymes may have an EC number and be well-characterized (their reactants and products are known), but not have a known associated amino acid sequence or genetic sequence (“orphan enzymes”). In such cases, the prediction engine 109 may bioprospect the orphan enzymes to predict their amino acid sequences, and, ultimately, their genetic sequences, so that the newly-sequenced enzymes may be engineered into the host cell to catalyze one or more reactions. The prediction engine 109 may then designate the reactions corresponding to the newly-sequenced enzymes as members of the filtered reaction data. In embodiments, the prediction engine 109 bioprospects the orphan enzymes using techniques known in the art. For example, one team determined the amino acid sequences for a small number of orphan enzymes by applying mass-spectrometry based analysis and computational methods (including sequence similarity networks and operon context analysis) to identify sequences. The team then used the newly determined sequences to more accurately predict the catalytic function of many more previously uncharacterized or misannotated proteins. Ramkissoon K R, et al. (2013) Rapid Identification of Sequences for Orphan Enzymes to Power Accurate Protein Annotation, PLoS ONE 8(12): e84508. doi:10.1371/journal.pone.0084508; see also Shearer A G, et al. (2014) Finding Sequences for over 270 Orphan Enzymes. PLoS ONE 9(5): e97250. doi:10.1371/journal.pone.0097250; Yamada T, et al., Prediction and identification of sequences coding for orphan enzymes using genomic and metagenomic neighbours genomic and metagenomic neighbours, Molecular Systems Biology 8:581, all three of which are incorporated by reference in their entirety herein.

Genome engineering. Embodiments of the disclosure identify biological sequences that enable functions in a host cell, and enable the host cell to use an identified biological sequence (e.g., by engineering the sequence into the host cell genome) to produce molecules. The bioreachable prediction tool may provide the list of bioreachable candidate molecules (viable target molecules) to a chemist, materials scientist or the like, who may be a third party such as a customer. Based upon their choice of target molecules, the user may instruct the tool to provide, to a gene manufacturing system, indications of the genetic sequences for the enzymes or other catalysts used to catalyze the reactions in the reaction pathways leading to each selected target molecule. The gene manufacturing system may then embody (through, e.g., insertion, replacement, deletion) the indicated genetic sequences into the genome of the host, to thereby produce an engineered genome for manufacture of the viable target molecules. In embodiments, the gene manufacturing system may be implemented using by systems and techniques known in the art, or by the factory 210 described in pending U.S. patent application Ser. No. 15/140,296, filed Apr. 27, 2016, published Nov. 2, 2017, entitled “Microbial Strain Design System and Methods for Improved Large Scale Production of Engineered Nucleotide Sequences,” incorporated by reference in its entirety herein. As described in that application, the gene manufacturing system may employ known techniques such as the Gibson and Golden Gate assembly protocols to assemble DNA sequences based upon input designs. The DNA constructs are typically circularized to form plasmids for insertion into a base strain. In the gene manufacturing system, the base strain is prepared to receive the assembled plasmid, which is then inserted. Input information may include techniques to employ during beginning, intermediate and final stages of manufacture. For example, many laboratory protocols include a PCR amplification step that requires a template sequence and two primer sequences. As is known in the art, the gene manufacturing system may be implemented partially or wholly using robotic automation. In embodiments, in addition to or as a substitute for embodying genetic sequences into the host, the prediction engine 109 provides to the factory an indication of one or more catalysts for the factory to introduce the one or more catalysts into the growth medium of the host cell for production of the target molecule.

Production of product of interest. Embodiments of the disclosure use well-known techniques to produce a viable target molecule or other product of interest from a base strain having a native or engineered genome. According to embodiments of the disclosure, the organism is transferred to a bioreactor containing feedstock for fermentation. Under controlled conditions, the organism ferments to produce a desired product of interest (e.g., small molecule, peptide, synthetic compound, fuel, alcohol) based upon the assembled DNA.

Different types of microbes can function as platform organisms in industrial biotechnology, including bacteria and yeasts fermenting sugar compounds into end-products, as well as microalgae via photosynthesis (phototrophic algae) or fermentation (heterotrophic algae).

The bacteria or other cells can be cultured in conventional nutrient media modified as appropriate for desired biosynthetic reactions or selections. Culture conditions, such as temperature, pH and the like, are those suitable for use with the host cell selected for expression, and will be apparent to those skilled in the art. Many references are available for the culture and production of cells, including cells of bacterial, plant, animal (including mammalian) and archaebacterial origin. See e.g., Sambrook, Ausubel (all supra), as well as Berger, Guide to Molecular Cloning Techniques, Methods in Enzymology volume 152 Academic Press, Inc., San Diego, Calif.; and Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W. H. Freeman and Company; and Ricciardelle et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue from Sigma-Aldrich, Inc (St Louis, Mo.) (“Sigma-LSRCCC”) and, for example, The Plant Culture Catalogue and supplement also from Sigma-Aldrich, Inc (St Louis, Mo.) (“Sigma-PCCS”), all of which are incorporated herein by reference.

The culture medium to be used should in a suitable manner satisfy the demands of the respective strains. Descriptions of culture media for various microorganisms are present in the “Manual of Methods for General Bacteriology” of the American Society for Bacteriology (Washington D.C., USA, 1981), incorporated by reference herein.

The synthesized cells may be cultured continuously, or discontinuously in a batch process (batch cultivation) or in a fed-batch or repeated fed-batch process for the purpose of producing the desired organic compound. A summary of a general nature about known cultivation methods is available in the textbook by Chmiel (Bioprozeßtechnik. 1: Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)), all of which are incorporated by reference herein.

Classical batch fermentation is a closed system, wherein the composition of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. Continuous fermentation generally maintains the cultures at a constant high density where cells are primarily in log phase growth. Continuous fermentation generally maintains the cultures at a stationary or late log/stationary, phase growth. Continuous fermentation systems strive to maintain steady state growth conditions.

Methods for modulating nutrients and growth factors for continuous fermentation processes as well as techniques for maximizing the rate of product formation are well known in the art of industrial microbiology.

For example, a non-limiting list of carbon sources for cellular cultures include, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

A non-limiting list of the nitrogen sources include, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

A non-limiting list of the possible phosphorus sources include, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate.

Finally, essential growth factors such as amino acids, for example homoserine and vitamins, for example thiamine, biotin or pantothenic acid, may be employed in addition to the abovementioned substances.

In some embodiments, the pH of the culture can be controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

The cultures may include an anti-foaming agent such as, for example, fatty acid polyglycol esters. The cultures may be modified to stabilize the plasmids of the cultures by adding suitable selective substances such as, for example, antibiotics.

The cultures may be carried out under aerobic or anaerobic conditions. In order to maintain aerobic conditions, oxygen or oxygen-containing gas mixtures such as, for example, air, are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation may be continued until an amount of the desired product of interest (e.g. an organic-chemical compound) sufficient for recovery has formed. This aim can normally be achieved within 10 hours to 160 hours. In continuous processes, longer cultivation times are possible. The activity of the microorganisms results in a concentration (accumulation) of the product of interest in the fermentation medium and/or in the cells of said microorganisms.

### Pathway Prediction Examples

The prediction engine 109 may predict every pathway of reactions employing catalysts likely available to catalyze the reactions in the pathways or be engineered into the host to reach a target molecule, according to embodiments of the disclosure. The prediction engine 109 may also be used to select from among the predicted pathways to attempt manufacturing of the molecule based on qualitative information or quantitative information such as a score that may be generated by the prediction engine 109.

### Reaction Labels and Categories

Reaction sets can be filtered and labeled as described elsewhere in this patent. For example, reactions can be labeled as “sequence relaxed,” to indicate they are likely to have gene sequences available, or they could be labeled as “characterized orphan” to indicate that genes exist in nature, but need to be experimentally characterized. Reactions can similarly be labeled to reflect their mass and energy balance, or other traits.

In addition, the bioreachable prediction tool may calculate in which direction a reaction is likely to operate based on thermodynamic data.

During processing of reactions to generate target molecules, the reaction annotation engine 107 can flag whether the production of a target molecule by a reaction happens in the thermodyanamically favorable direction or in the thermodynamically unfavorable direction.

These thermodynamic results and all of the other reaction labels can then be used by the reaction annotation engine 107 to tag the molecules and pedigrees produced by a given run of the bioreachable prediction tool. For example, a five-step pedigree that contains one thermodynamically unfavorable reaction and two reactions lacking known genes to produce enzymes to catalyze the reactions could be labeled as:

Path length: 5

Unfavorable reactions: 1

Reactions lacking genes: 2

These labels then may be used by the prediction engine 109 to score each reaction. They also can be used to sort and operate on subsections of output, and they provide a direct insight into the engineerability of a given molecule for a given host.

In the examples detailed below, the bioreachable prediction tool was used to identify target molecules and display predicted pathways that may be used to reach those target molecules.

Thermodynamic data that was incorporated into pathway production and evaluation was generated using the group contribution method, but could also have been derived from any number of metabolic databases.

The prediction engine 109 may assign to each potential pathway an associated score created using the scoring method described herein. These scores can be used to inform decisions about which pathway variation to attempt to engineer to make the target molecule.

In embodiments, the prediction engine 109 may start with an optimal score of 100 points and subtract points for pathway features that add difficulty or risk of design failure. For example, path length correlates with design risk, and the total score may be reduced as path length increases, e.g., the prediction engine 109 may subtract from the score one or more points for each additional step in path length.

Tyramine

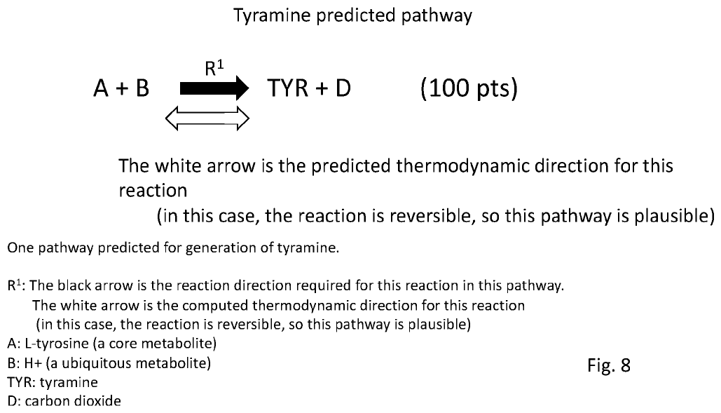

FIG. 8 illustrates a pathway identified by the prediction engine 109 to produce tyramine, according to embodiments of the disclosure. In the case of tyramine, a single pathway consisting of one reaction step (R¹) was predicted. The pathway shown depends on a reaction that is calculated based on thermodynamic data to be reversible, meaning it can operate in the direction required to generate tyramine.

In the pathway figures, a black arrow represents the reaction direction required for that reaction in the pathway to produce the desired molecule (here, tyramine). A white arrow represents the calculated thermodynamic direction for a reaction. When the required and calculated reaction directions match, the pathway is plausible.

This single pathway scores 100 points by the metric described elsewhere.

(S)-2,3,4,5-tetrahydrodipicolinate (THDP)

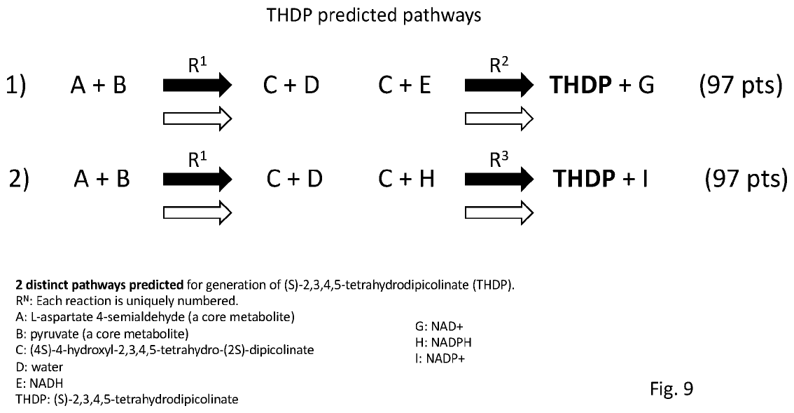

As shown in FIG. 9, the bioreachable prediction tool predicted two possible two-step pathways to generate THDP, according to embodiments of the disclosure. Both pathways achieve the same score of 97 points in these embodiments.

The pathways share the same first reaction (R¹) and differ at the second reaction (R² or R³). In this case, these reactions differ in which form of reducing cofactor they use, e.g., NADH versus NADPH. Although the pathways score the same, this cofactor difference is relevant for engineering purposes, and thus is displayed in this embodiment of the bioreachable prediction tool to help guide design decisions. Typically, one cofactor (either NADH or NADPH) is far more abundantly present in each given host cell. Thus, in embodiments, one skilled in the art may select the pathway employing the more abundant cofactor to produce THDP. In other embodiments, the prediction engine 109 may retrieve from a database and consider information concerning the influence of cofactors on engineerability to compute the target molecule score, thereby obviating the need for human review of the pathway cofactors.

Example predicted pathways for hypothetical molecule “F”

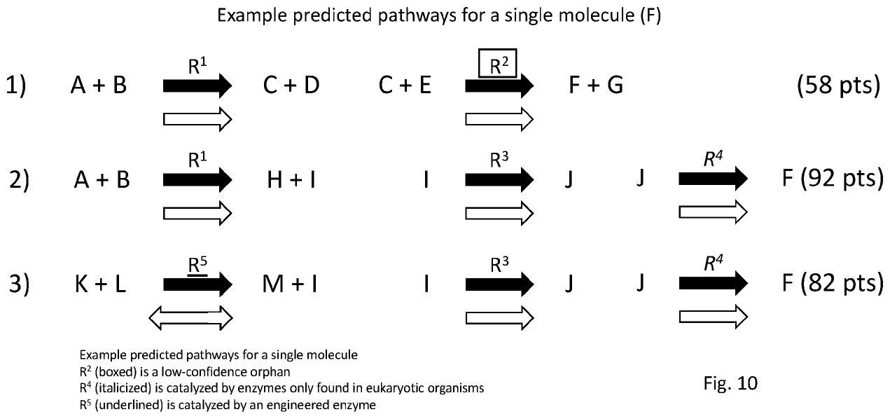

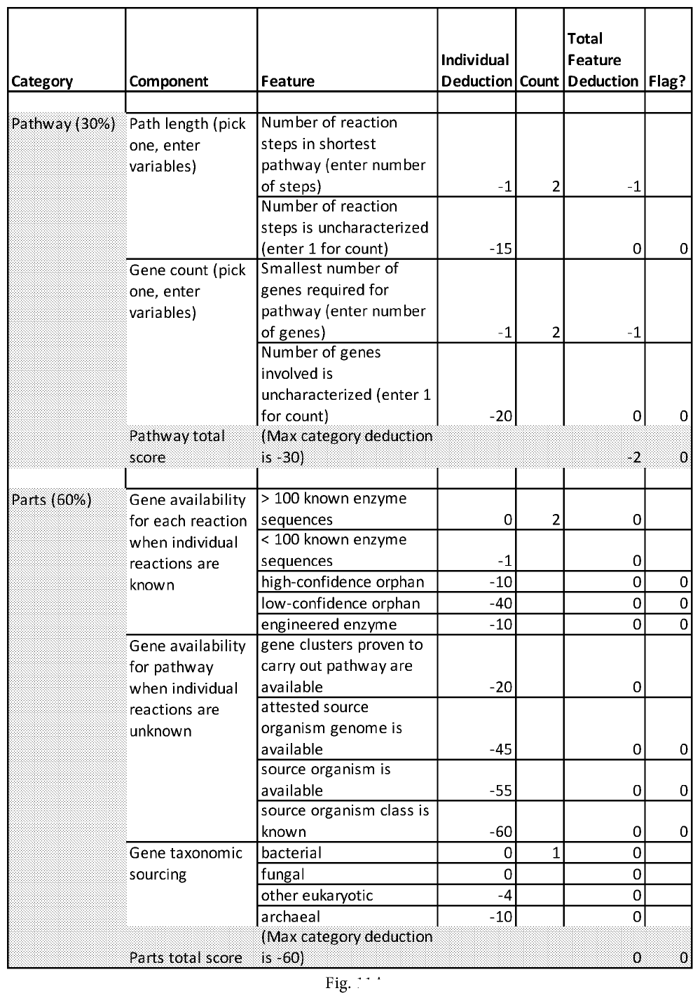

In another example, for a bioreachable molecule “F,” the bioreachable prediction tool has predicted three potential pathways, as illustrated in FIG. 10.

The first pathway is two steps long and includes a low-confidence orphan reaction (R²), leading to a score of 58 points. A low-confidence orphan reaction is a reaction catalyzed by an orphan enzyme for which it is unlikely that the corresponding DNA sequence is readily available without extensive, specific research work. Thus, many points are deducted for the orphan enzyme.

The second pathway is three steps long and includes one reaction with only eukaryotic genes available (R⁴), leading to a score of 92 points. Points are deducted because of overall pathway length and because of the limitation in sourcing genes for R⁴.

The third pathway is also three steps long and has two reactions (R³ and R⁴) in common with the other three-step reaction. It also has one reaction (R⁴) with only eukaryotic genes available and another reaction (R⁵) that requires an engineered enzyme, leading to a score of 82 points. In addition, this pathway has an alternate set of starting core metabolites (K+L instead of A+B) which has no impact on the pathway score, but is a consideration when deciding on which pathway is a best fit for the specific host and application.

In this example, the scoring output from the bioreachable prediction tool's prediction engine 109 provides critical engineering information beyond simple path length. Despite intuition that the shortest pathway (#1) might be best, information collected by the annotation engine 107 about each reaction and by the bioreachable prediction tool during filtering or processing show that the longer pathways (#2 and #3) might be more feasible to engineer. For example, the reaction annotation engine 107 may determine that catalysts for some reactions are only available in high-risk categories (e.g. low-confidence orphans, engineered enzymes), and the prediction engine 109 may determine that the short pathway depends on these high-risk categories whereas the long pathway does not, which may show that a longer pathway may be more feasible to engineer.

Tetrahydrodipicolinate Scoring Table

According to embodiments of the disclosure, the prediction engine 109 uses the information it generates to score the difficulty of producing target molecules. (Conversely, the score may be viewed as indicating the ease of producing molecules.) This score is interchangeably referred to herein as “molecule score,” “target molecule score,” or “overall pathway score.”

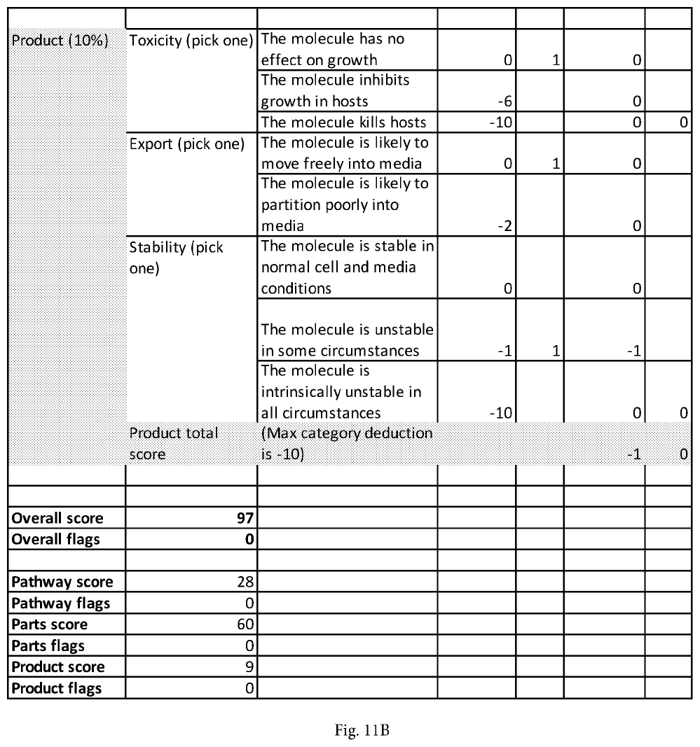

As an example, FIGS. 11A and 11B together provide a table illustrating how the prediction engine 109 may score the production of tetrahydrodipicolinate (THDP). In embodiments, the overall pathway scoring process may be broken down by components such as pathway score, parts score, and product score, weighted, e.g., as 30%, 60%, 10%, as shown in the table. The evaluation data shown was generated during the process of predicting pathways to the molecule (S)-2,3,4,5-tetrahydrodipicolinate (THDP).

Pathway component score represents the relative engineering feasibility of the pathway. In embodiments, it comprises two elements:

Path length—The number of reaction steps in the pathway. This is tallied as an intrinsic part of bioreachable prediction by the prediction engine 109, according to embodiments of the disclosure.

Gene count—The number of genes predicted to be required for the pathway. This is identified by querying databases as part of reaction filtering by the reaction annotation engine 107.

Since reactions and enzymes are not always in a 1:1 relationship (for example, a single reaction is sometimes catalyzed by a two-part enzyme, requiring two genes), the prediction engine 109 may factor both elements into the predicted difficulty of engineering the pathway.

In both pedigrees predicted by the bioreachable prediction tool, as shown in

FIG. 9 THDP requires a two-step pathway in the desired host cell. This yields an appropriate score deduction based on the modest increase in difficulty of a 2- versus 1-step pathway.

In this case, the number of genes per pathway reaction step (identifiable via the same evaluation process that determines if a reaction is likely to have genes at all) also yields a modest penalty.

Parts Component Score

The Parts score represents the relative engineering feasibility of the individual pathway parts. In embodiments, it is based on the predicted difficulty in finding the parts (e.g., genes) required to engineer a catalyst into a host for the reactions in the pathway that is being evaluated.

In embodiments, the possible features that can impact the ability to find parts include:

>100 known enzyme sequences—100 or more sequences found for the reaction during the reaction filtering step (e.g., 100 or more amino acid sequences indicated in at least one database corresponding to enzymes for catalyzing the reaction)

<100 known enzyme sequences—enzyme sequences were found, but fewer than 100 were identified during the reaction filtering step

high-confidence orphan/low-confidence orphan—no enzyme sequences were found in public databases during the reaction filtering step, but associated evidence was found that suggested those sequences would be relatively easy (high-confidence) or hard (low-confidence) to identify

engineered enzyme—the only enzymes linked to this reaction during the reaction filtering step were engineered to carry out the reaction (this data can be found in database searches). This typically refers to natural enzymes that have been mutated to catalyze a reaction different from the reaction they naturally catalyze. These engineered enzymes can be difficult to use in novel pathways as they may be limited to one or a few sequences from a limited range of donor cells. Such engineered enzymes can be found in public databases such as BRENDA

gene taxonomic sourcing—also identified during the reaction filtering step (assuming enzyme sequences were found); this component classifies a candidate bioreachable molecule by the “worst case” (biggest penalty) among the reactions in the predicted pathway for that molecule; penalties are based on empirical data to date on the difficulty of expressing enzymes from the indicated sources in industrial platform cells

gene availability for pathway when individual reactions are unknown—in some cases pathways are defined using stand-in reactions in the dataset, and these reactions can be programmatically linked to individual gene clusters or cells; pathways in which individual reactions are unknown represent a significant increase in engineering risk and difficulty and thus a large penalty is assigned

These feature elements are all identified by the reaction annotation engine 107, as information is accumulated about the presence, absence, and abundance of sequence data for enzymes that catalyze each reaction.

In the case of THDP, genes are abundantly present for both pathway reactions, yielding no penalty. If instead, for example, one of the reactions were catalyzed by a low-confidence orphan, THDP would have accrued a significant penalty.

Product Component Score

The Product score is the smallest overall contributor to the target molecule score, in embodiments of the disclosure. The product score represents factors that influence the difficulty in sustaining the product in the cell, exporting it from the cell, and maintaining it in media. In embodiments, it represents an evaluation of the molecule's expected toxicity, exportability, and stability. The specific features described in this embodiment include:

Toxicity—The degree to which the molecule might be expected to be toxic to one or more host cells. This information can be derived from querying antimicrobial databases (or other databases that collect toxicity information on the general category of host cells).

Export—Predicted by querying chemical databases for partition coefficient data, or by querying internal experimental data.

Stability—Stability issues are identified by querying chemical databases.

Score Summary

The bottom of the table summarizes the overall score and the category scores. It also highlights any flags—areas that require particular derisking for pathway engineering. THDP happens to have no flags. An example flag would be if a pathway is missing one or more genes for its reaction steps (e.g., high- or low-confidence orphans).

### Algorithmic Enzyme Selection

Overview

Embodiments of the disclosure that include algorithmic biological sequence selection provide an algorithmic, computer-implemented approach to select enzymes as candidates for catalyzing a reaction. This approach substantially reduces the time required to determine optimal enzymes and eliminates human error. It also enables continuous improvement of the tool's prediction accuracy via refinement of its predictive models based on the empirical data generated as a result of experimental validation of the sets of selected sequences.

Because of the ability to handle enormous data sets, embodiments employing algorithmic biological sequence selection may cause an exponential increase in potential candidate sequences. Embodiments of the disclosure address this issue by performing clustering or alternative path elimination (or both) to refine the selection of candidate sequences while maintaining the diversity of the sequence space.

Moreover, embodiments of the disclosure enable the identification of sequences that are statistically more similar to the desired function than manual approaches that rely on the functional human annotation of sequences.

More generally, embodiments of the disclosure may select sequences for enabling the performance of a desired function in a host cell. In addition to enzymes, such sequences may include, for example, transporters, transcription factors, and nucleic acid sequences that code for proteins such as enzymes for catalyzing reactions. In addition to an enzymatic reaction, functions may include facilitation or regulation of cellular processes such as gene transcription/translation, transport of molecules across membranes, and stabilization or degradation of molecules.

Embodiments of the disclosure identify candidate biological sequences for enabling a function in a host cell based upon sequences that are known or believed to enable the same or a similar function in different cells. The cells may, for example, be found in different species. In other cases, different sequences that carry out the same function in the same species, however, may exhibit different attributes that a scientist would find desirable for one purpose but not another.

Glossary

A biological sequence is a sequence of nucleotides or amino acids.

To clarify, unless otherwise indicated herein, the term “molecule” refers to a type of molecule (e.g., a particular type of protein molecule), and not to an individual isolated molecule.

Similarly, to clarify, unless otherwise indicated herein, the term “cell” refers to a type of cell, and not to an individual isolated cell.

Unless otherwise indicated herein, the terms “actual bioreachable” molecule, “actually bioreachable” molecule, and “bioreachable” molecule are used interchangeably herein to refer to a molecule that can be produced in vivo, in vitro, or otherwise using one or more biological processes (e.g., bio-catalysis, transcription, translation).

Unless otherwise indicated herein, the term “candidate bioreachable molecule” or, interchangeably, “bioreachable candidate molecule,” refers to a molecule that is likely a bioreachable molecule. In embodiments, a candidate bioreachable molecule may be a molecule predicted to be a bioreachable molecule (e.g., in one or more given host cells) based on a set of starting metabolic reactions and metabolites. In embodiments, a candidate bioreachable molecule may likely be a bioreachable molecule that has not yet been confirmed to be bioreachable. In embodiments, a candidate bioreachable molecule may be a molecule stored in a database (e.g., database 110) for candidate or actual bioreachable molecules, but that has not yet been identified in the database as actually bioreachable. In embodiments, a candidate bioreachable molecule is a molecule with evidence (e.g., identified in a database) of being synthesized or isolated in a biological system (e.g., a single organism, or a consortium of multiple organisms or tissue types). A bioreachable candidate molecule may be a molecule suspected to be bioreachable because, for example, it has been predicted to be a viable target molecule using embodiments that are described in sections above. In embodiments, the term “candidate bioreachable molecules” includes the viable target molecules predicted by embodiments of the disclosure described above.

The term “putative bioreachable molecule” shall refer to an actual bioreachable molecule or a candidate bioreachable molecule.

Operation

In embodiments of the disclosure, the prediction engine 109 includes program code for identifying a candidate biological sequence for enabling a function in a host cell. The prediction engine 109 may: access a predictive model that associates a plurality of biological sequences with one or more functions; predict, using the predictive model, that one or more candidate sequences of the plurality of biological sequences enable a desired function in the host cell; and classify candidate sequences that satisfy a confidence threshold as filtered candidate sequences. In embodiments, the biological sequences are enzymes for catalyzing reactions (the function being the enzyme-catalyzed reaction). The prediction engine 109 may provide to a gene manufacturing system information concerning a first filtered candidate sequence, so that the gene manufacturing system may use the first filtered candidate sequence to produce a molecule, which may, for example, be a bioreachable molecule.

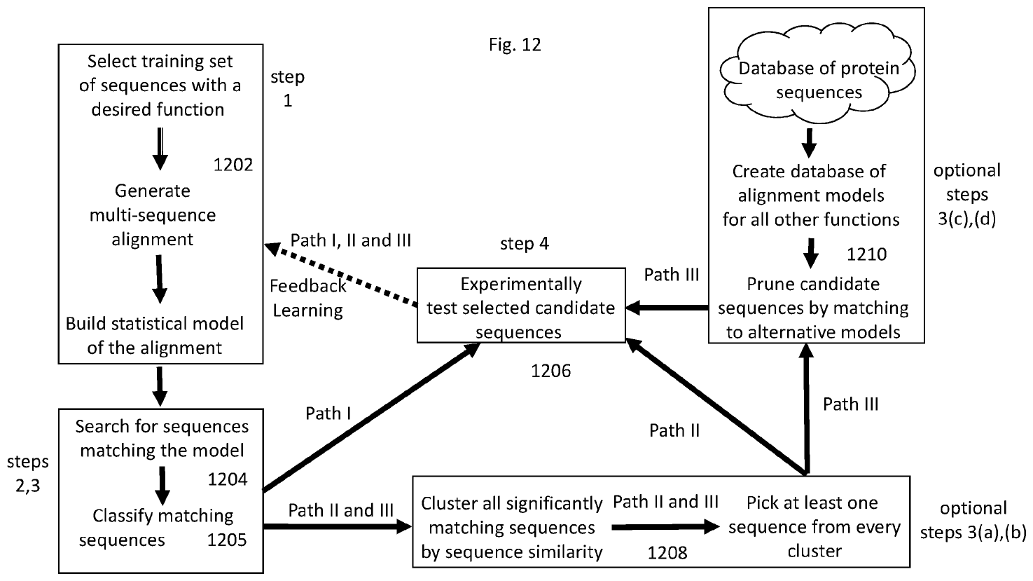

FIG. 12 is a flow diagram illustrating the operation of embodiments of the disclosure. Unless otherwise indicated, these operations may be performed by software residing in the prediction engine 109. Although the description below concerns the identification of enzyme amino acid sequences, the same approach may be used to identify other sequences, as noted below.

According to embodiments of the disclosure, the prediction engine 109 may perform the following operations:

Step 1 1202: obtaining the predictive model

The prediction engine 109 may generate (or retrieve from an internal or external database) one or models trained on instances of enzymes physically verified, or predicted with a high degree of confidence, to carry out the desired function. Examples of functions are: enzymatic activity such as tyrosine decarboxylase, which is an enzyme that catalyzes the conversion of tyrosine to tyramine; and alpha-amylase, which is an enzyme that catalyzes the hydrolysis of alpha-bonds in complex polysaccharides.

Instead of enzymes, embodiments of the disclosure may identify nucleic acid sequences that code for enzymes of interest. Further, functions represented by such models are not limited to enzymes of metabolic reactions, however, and may also, for example, refer to functions, such as DNA helicases, which are responsible for separating two strands of DNA or proteins, and other non-catalytic types of functions such as transcription factors, transporters, structural proteins, as well as nucleotide sequences that are not translated into peptides such as transfer RNAs, and small non-coding RNAs. In addition, one or multiple models can be generated for each functional activity that abstracts diversified information such as phylogeny, orthology, sequence similarity, enzyme subunits, and protein morphology.

The term “models” here includes but is not limited to statistical models such as Hidden Markov Models (HMMs), dynamic Bayesian networks, artificial neural networks (ANNs) including recurrent neural networks such as those based on Long Short Term Memory Models (LSTM) as well as derivatives and generalizations thereof, and other machine learning-based models.

As an example of a predictive model, for step 1, the prediction engine 109 may rely on HMM, which is a statistical model of multiple sequence alignments (MSAs). In bioinformatics, a sequence alignment is a way of arranging the sequences such as DNA, RNA, or protein, to identify regions of similarity that may be a consequence of functional, structural, and/or evolutionary relationships among the sequences. In evolutionary biology, conserved sequences are similar or identical sequences in nucleic acids (DNA and RNA) or proteins across species (orthologous sequences) or within a genome (paralogous sequences). Conservation indicates that a sequence has been maintained by natural selection. Amino acid sequences can be conserved to maintain the structure or function of a protein or domain.

As an example of finding a protein amino acid sequence for a reaction (function), which may be part of the reaction pathway output of embodiments above, the prediction engine 109 may retrieve from database 110 a training set of enzymes that catalyze the reaction. Each enzyme may be found in a different species. However, not every amino acid in the enzymes is important to performing the function. The observed frequency with which an amino acid occupies the same position in different enzyme sequences that perform the same function (the degree to which the amino acid is “conserved”) correlates to the likelihood that the amino acid enables performance of that function. This is the basis for using an MSA to identify other enzyme sequences for performing a desired function. The prediction engine 109 employing an MSA model provides the output sequences along with a measure of the degree of confidence (based on the conservation of the sequences) that a sequence enables the desired function.

Conserved sequences may be identified by homology search, using tools such as BLAST, HMMER and Infernal. Homology search tools may take an individual nucleic acid or protein sequence as input, or use statistical models generated from multiple sequence alignments of known related sequences. Statistical models, such as profile-HMMs, and RNA covariance models which also incorporate structural information, can be helpful when searching for more distantly related sequences. Input sequences are then aligned against a database of sequences from related individuals or other species. The resulting alignments are then scored based on the number of matching amino acids or bases, and the number of gaps or deletions generated by the alignment. Acceptable conservative substitutions may be identified using substitution matrices such as PAM and BLOSUM. Highly scoring alignments are assumed to be from homologous sequences. The conservation of a sequence may then be inferred by detection of highly similar homologs over a broad phylogenetic range.

Identifying conserved sequences can be used to discover and predict functions of sequences such as proteins and genes. Conserved sequences with a known function, such as protein domains or motifs, can also be used to predict the function of a sequence. Databases of conserved protein domains or motifs such as Pfam and the Conserved Domain Database can be used to annotate functional domains or motifs of predicted proteins.

### Example Inputs and Outputs

Input step 1: an enzymatic activity/reaction from a predicted pathway/pedigree, such as “tyrosine decarboxylase,” which may be represented by the chemical equation “L−Tyrosine<=> Tyramine+CO2,” and a training set of sequences that are believed to have this enzymatic activity/catalyze this reaction (e.g., based on scientific publications, experimental data from a public or internal database or a computational prediction based on homology to sequences with experimental evidence of the required activity).

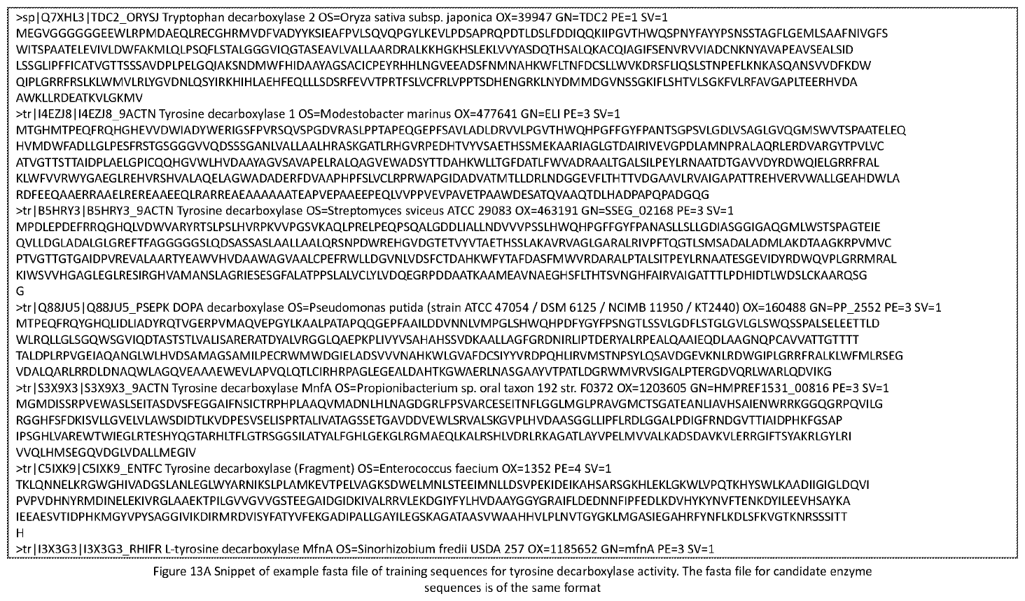

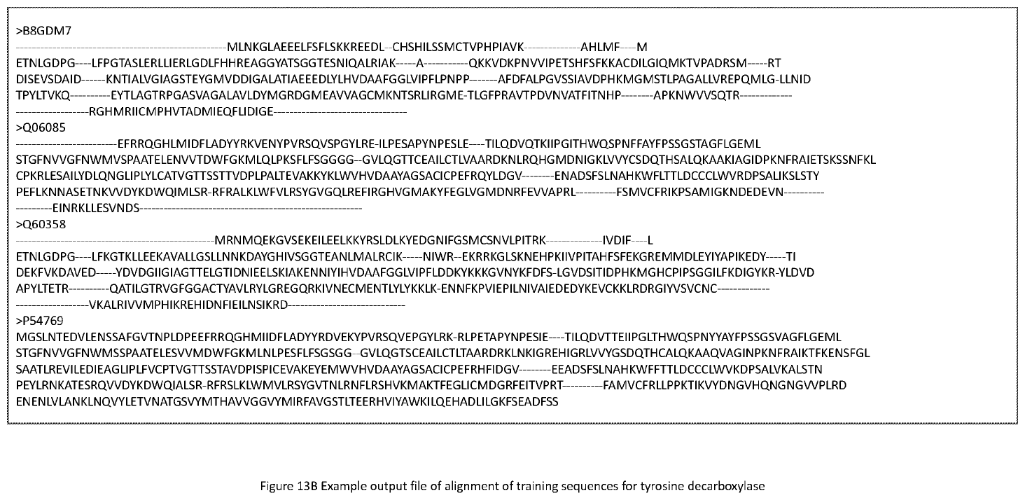

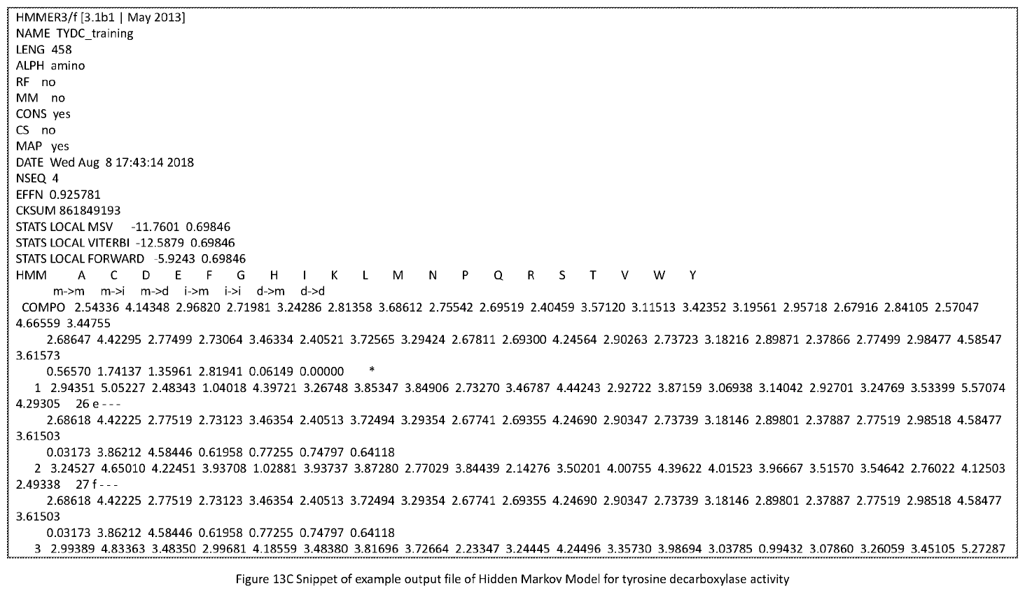

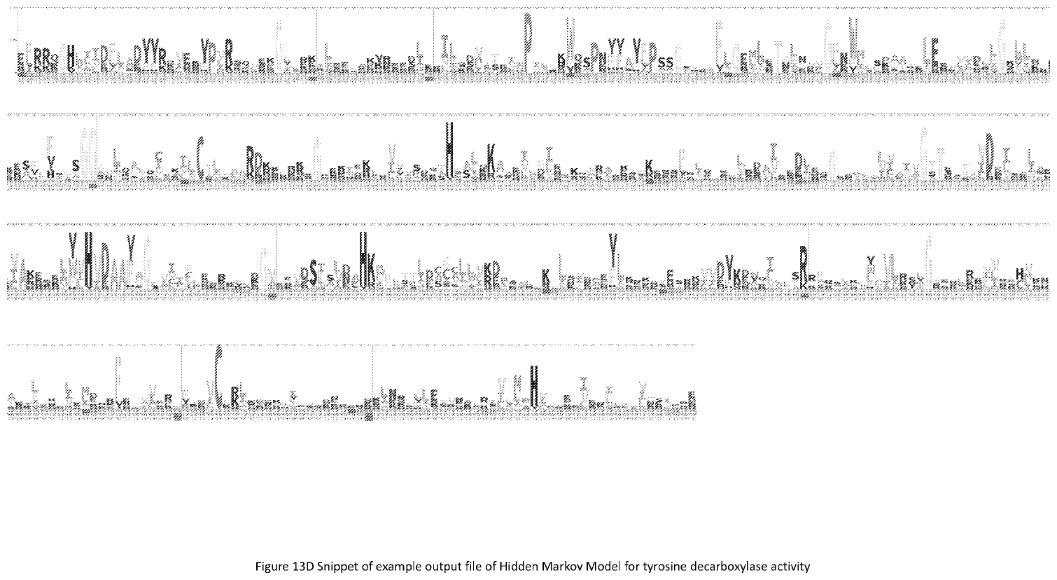

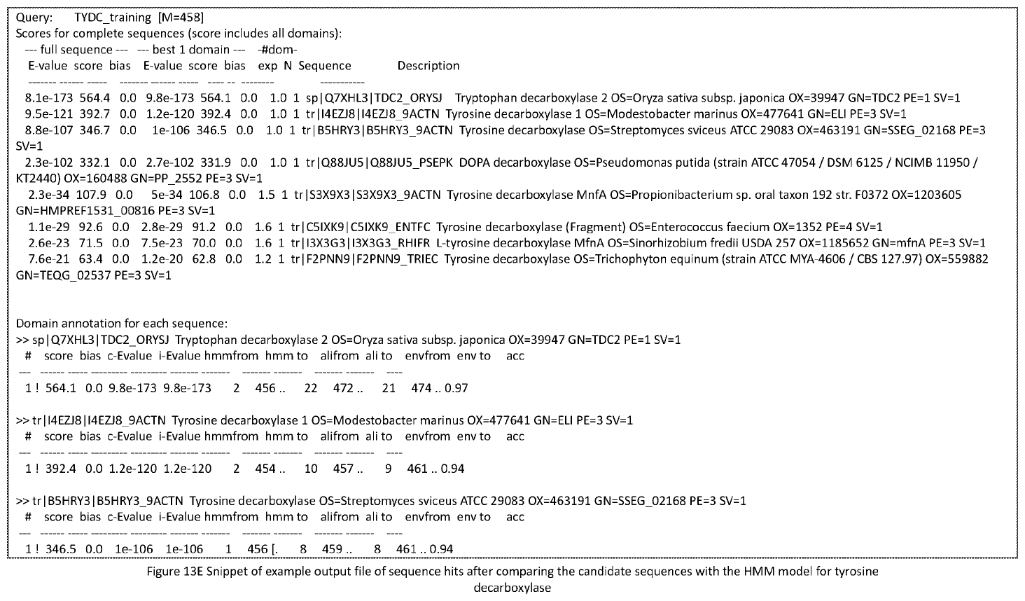

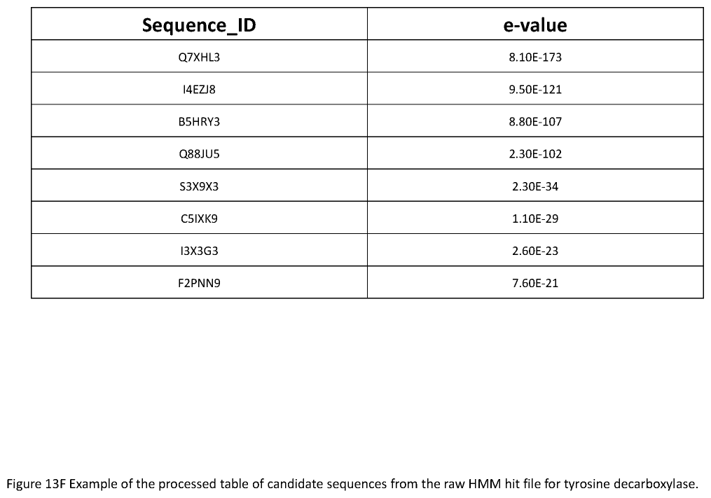

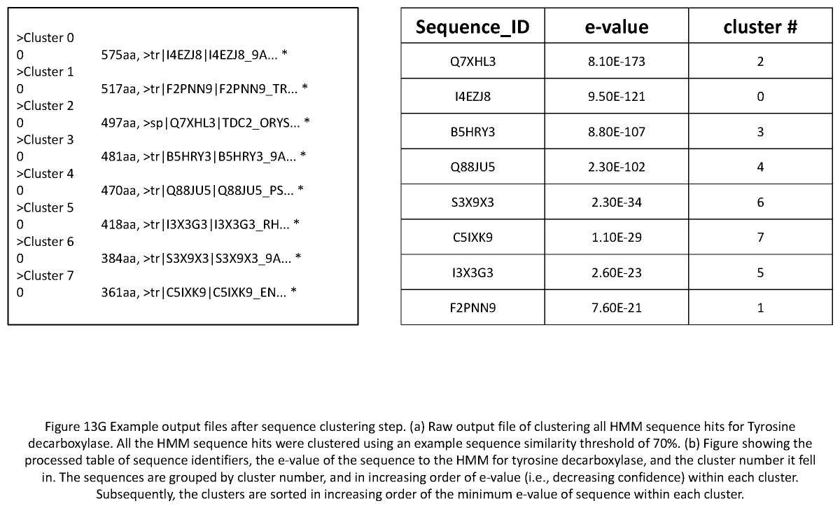

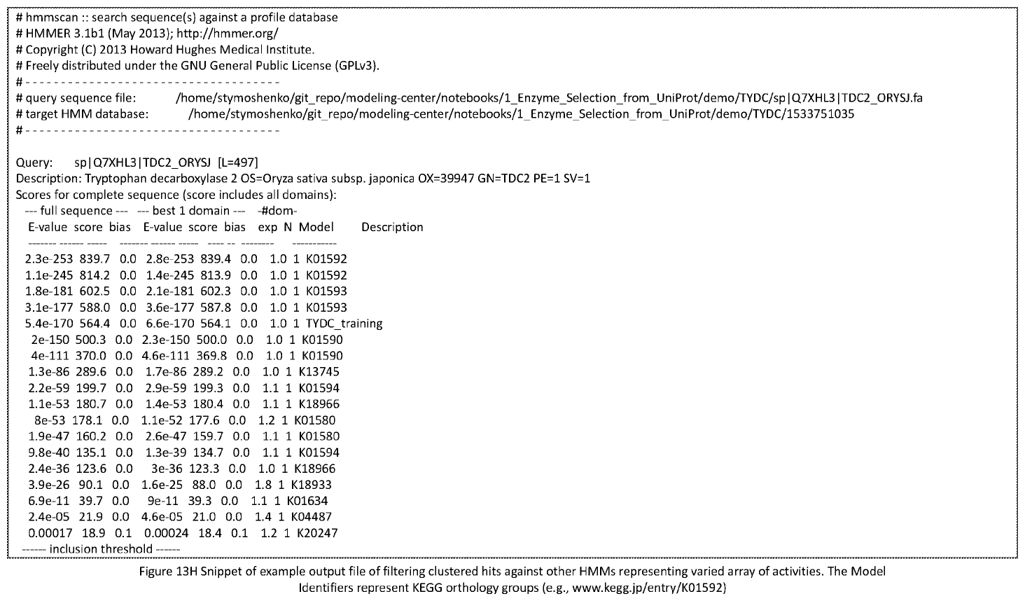

FIGS. 13A-H illustrate a prophetic example of identifying at least one sequence to enable tyrosine decarboxylase activity using the HMMER tool, according to embodiments of the disclosure. One of ordinary skill in the art would understand how to interpret these figures, especially in view of Eddy, et al., HMMER User's Guide: Biological sequence analysis using profile hidden Markov models, Version 3.1b2; February 2015, incorporated by reference herein in its entirety.

FIG. 13A illustrates a snippet of an example FASTA file containing a training set of enzymes that catalyze for tyrosine decarboxylase activity. The file contains the amino acid sequences of the training set of enzymes encoding for the reaction activity. Note that the annotations in the file indicate activity other than tyrosine decarboxylase, such as tryptophan decarboxylase, because the displayed annotations were derived from a commercially available database. However, embodiments of the disclosure determined that such sequences, in fact, enabled tyrosine decarboxylase activity. Thus, embodiments of disclosure enable correct recordation of annotations in otherwise incorrect publicly available databases.

Output step 1: multi-sequence alignment(s) of the sequences present in the training set and a model (or multiple models) representative of this alignment, including an indicator of the degree of confidence that a unit within the sequence (e.g., an amino acid) is related to the desired function (e.g., expectation value, probability that the unit is conserved at a given position within the sequence). FIG. 13B shows snippet of an output file showing such a multi-sequence alignment of the training set of enzymes encoding for a tyrosine decarboxylase reaction. An identifier (e.g., B8GDM7) following the “>” sign identifies an enzyme sequence, and the text below shows the corresponding sequence. In this example, spaces, as indicated by “−” in the amino acid sequences, indicate positions where a particular enzyme sequence does not align with the consensus alignment of all enzymes in the training set of enzymes. The consensus alignment is determined by optimal subsequences that are conserved, through similarity and/or identity, across all the sequences in the training set of enzymes.

FIG. 13C shows a snippet of an output file of a Hidden Markov Model (using the HMMER tool) constructed from the multi-sequence alignment file shown in FIG. 13B, from which a skilled artisan can determine the degree of confidence that an amino acid within the sequence is related to the desired tyrosine decarboxylase activity (function). FIG. 13D shows a pictorial representation of the same statistical model for tyrosine decarboxylase activity, where the height of the each amino acid annotation represents the propensity of that particular amino acid in that position (represented on the x axis) to be related to the desired function of the overall enzyme.

Step 2 1204: matching database of sequences to model

The prediction engine 109 may perform a search for candidate sequences for enabling the function of interest using the model(s) trained in step 1, by comparing every sequence in a source database (such as Uniprot, KEGG, NCBI, JGI GOLD or a proprietary database of nucleotide or protein sequences) to the model(s) generated in step 1. Examples of the tools that could be used for this process is HMMsearch, HMMscan, or Recurrent Neural Networks designed for search by LSTM models.

### Example Inputs and Outputs

Input step 2: the model(s) trained on the trusted set(s) of sequences with the desired function and a search database of sequences.

Output step 2: due to the size of the source databases, the prediction engine 109 may output a set of sequences ranging from a few to 100,000s (for just one reaction) that significantly match (with a high probablility score) to the model(s) produced in the step 1. FIG. 13E shows a snippet of example output file of sequence hits after comparing the candidate sequences with the HMM model for tyrosine decarboxylase. In this example file, the confidence of a particular enzyme sequence from a database matching to the HMM of tyrosine decarboxylase is enumerated by the E-value metric. The lower the E-value of an enzyme, the higher the statistical confidence of a match to the model.

FIG. 13F shows an example of the processed table of candidate sequences from the raw output file for FIG. 13E that extracts the identifier of the sequence from the search database and the E-value of the match to the tyrosine decarboxylase HMM model sorted in ascending order of E-value. In this example, the enzyme sequence Q7XHL3 has the lowest E-value, and thus is ranked as the amino acid sequence most likely to enable tyrosine decarboxylase activity.

Embodiments of the disclosure provide further refinements to reduce the size of this potentially enormous data set.

Step 3 1205: filtering matching sequences

The prediction engine 109 may classify the candidate sequences from step 2 based on threshold parameters (e.g., minimal probability score such as expect value (E-value) or significance threshold) that may be determined by the user or another based on the intended purpose and trade-offs between precision and scope of the search. For example, assume step 2 results in a large number of sequences that enable the desired function with low degrees of confidence. In such cases, a user may adjust a first confidence threshold so that the prediction engine 109 eliminates sequences that do not satisfy that first threshold to result in a more manageable number of candidate sequences with higher confidence. The candidate sequences that satisfy the first confidence threshold (surviving step 3) may be referred to as “filtered candidate sequences” if the workflow follows Path I, shown in FIG. 12 and described below. If Path II or Path III is taken, then the candidate sequences that enter step 4 from optional step 3(b) or 3(d), respectively, may be referred to as “filtered candidate sequences.”

For example, depending on the size of the training set, size of the sequence database, and number of candidate sequences found at the step 2, as well as other factors, a user may set the minimal degree of confidence, e.g. expect-value, as permissive as 1E-10* or higher (to broaden the scope of the search by sacrificing precision), or, conversely, as strict as 1E-50** or lower to increase the precision with the caveat of a reduced scope.

* estimated one out of ten billion (10¹⁰) randomly-generated sequences would be a better match to the given model than the candidate sequence with the e-value 1E-10

** estimated one out of 10⁵⁰ randomly-generated sequences would be a better match to the given model than the candidate sequence with the e-value 1E-50.

### Example Inputs and Outputs

Input step 3: One or more sequences that match the model(s) representing the function of interest

Output Step 3: A subset of (filtered) candidate sequences that match the model(s) representing the function of interest and satisfy a user-defined minimal, first degree of confidence threshold.

Step 4 1206: refining predictive model

The candidate sequences that satisfy the first confidence threshold in step 3 may be synthesized and tested to ascertain empirically if they catalyze the desired function as predicted by the model. (The same operations may be performed on the candidate sequences resulting from optional Paths II and III, which are described below.) This test can be performed as an in vitro enzyme assay, or via incorporation of the sequences into host(s) through, but not limited to, chromosomal integration or replicated plasmids. For those sequences that produced the desired function under the particular experimental conditions, the prediction engine 109 may record the result in the model database (e.g., database 110). For those sequences where the desired function was not detectable, prediction engine 109 may also record that result in the database 110. The prediction engine 109 may use these records to expand/refine the set of training sequences for the model(s) representing this function as the “positive” and “negative” training set/examples.

According to embodiments of the disclosure, the prediction engine 109 repeats steps 1-4 (and steps 3(a)-(d), to the extent those options are chosen) for each reaction (e.g., in a pathway leading to a specified putative bioreachable molecule), and stores the results in database 110.

A change in the experimental setting (such as a change in the host cell or growth media) may change the empirical outcomes. For example, not all sequences may produce the desired function in all possible conditions. The prediction engine 109 may record this result in the database 110 such that subsequent searches with the same combination of host and experimental conditions would exclude the negative examples.

The number of sequences chosen to be validated experimentally may be limited by available throughput. In a high-throughput factory-like setting, in principle, many sequences could be tested simultaneously for the same functionality. The “re-training,” via feedback loop, of the models based on positive and negative outcomes observed enhances the predictive power and precision of the models with every select-test-retrain cycle (illustrated as part of Paths I, II and III in FIG. 12). To this end, automated, high-throughput experiments can yield large and consistent training sets, thereby enabling retraining in a consistent manner that is robust to occasional errors and biological variability.

Example Inputs and Outputs

Input step 4: candidate sequences to be validated

Output step 4: recorded results of experimental validation in database to update predictive model

Optional steps 3(a) and 3(b) 1208: clustering

Referring to FIG. 12, steps 1, 2, 3 and 4 described above follow the arrows labeled with “Path I.” FIG. 12 also illustrates optional Paths II and III, which may be performed to further refine the filtered candidate sequences, according to embodiments of the disclosure. The candidate sequences resulting from Paths II and III, like those from Path I, are subject to step 4, according to embodiments of the disclosure.

Path II includes steps 3(a) and 3(b) 1208. In embodiments, the prediction engine 109 may (e.g., if the user elects) take additional steps 3(a) and 3(b) before step 4 to diversify the candidate sequences that satisfy the first confidence threshold.

Step 3(a) 1208: The predictive engine 109 may perform statistical clustering (based on, for example, sequence similarity, or t-Distributed Stochastic Neighbor Embedding) on the candidate sequences that satisfy the first confidence threshold. The prediction engine 109 may record which sequences are sufficiently similar to appear in the same cluster. For example, using the CD-HIT clustering algorithm, the prediction engine 109 may denote sequences as belonging to the same cluster if they exceed a 38%-99% sequence identity threshold. This value is a user-defined parameter that reflects the maximal degree of identity among the sequences, which a user allows to include in the final filtered set of candidates. In the left table, FIG. 13G shows a snippet of the raw output file resulting from clustering all HMM sequence hits for tyrosine decarboxylase. All the HMM sequence hits are clustered using an example sequence identity threshold of 70%. The figure shows a snippet of the file that lists the cluster number and the sequence identifiers of all the sequences that lie within that cluster. (In this snippet, the full list of sequence identifiers is truncated as indicated by the asterisks.) In this manner, a user can address the challenge of evenly exploring candidate sequences when their number exceeds the experimental capacity for testing all the candidates.

Optional step 3(b) 1208: selecting sequence(s) from the clusters

The prediction engine 109 may select one or more sequences from each cluster. The number of sequences selected may depend upon the number of clusters, which in turn depends on the user-defined sequence identity threshold as well as the overall “sequence diversity” within the set of candidate sequences prior to the clustering. Selection of a particular candidate sequence(s) from each cluster may be informed by the degree of confidence (e.g. the e-value of the match to the corresponding model). This ensures that not only a diversified set of candidates are selected for each function/reaction but also that the candidates with the highest likelihood of desired function are prioritized. FIG. 13G (right table) shows the example processed table output of sub-selected sequences where only the sequence with lowest e-value is selected from each cluster, after clustering step 3(a). The table shows the identifiers of those enzymes, the e-value of the sequence matching to the HMM for tyrosine decarboxylase, and the cluster number in which it fell, which is generated by parsing the output file in the left table of the figure. The right table shows the sorted sequences by increasing e-value (i.e., decreasing confidence).

Optional steps 3(c) and 3(d) 1208: eliminating candidate sequences that have affinity toward alternative functions

Path III includes steps 3(c) and 3(d) 1210. In embodiments, the prediction engine 109 may (e.g., if the user elects), take additional steps 3(c) and 3(d) before step 4 to reduce the likelihood that the candidate sequences that satisfy the first confidence threshold represent undesired functions. In embodiments, steps 3(c) and 3(d) may be chosen only if the confidence scores of the candidate sequences that satisfy the first confidence threshold are above or below a second threshold.

Optional step 3(c): creating data set of models for other functions

In embodiments, the prediction engine 109 may prepare a database of predictive models that represent all known functions for which such model(s) can be constructed, e.g., all KEGG orthology groups that are associated with at least one sequence that has been empirically observed to carry out a corresponding function.

Optional step 3(d): eliminating candidate sequences that have affinity toward alternative functions

In embodiments, the prediction engine 109 may prevent classification, as a filtered candidate sequence, of a candidate sequence that satisfies the first confidence threshold but that is more likely, within a given tolerance (e.g. between 0.5 and 1, where 1 represents no tolerance to the possibility of an alternative function), to enable a function different from the desired function. To do so, the prediction engine 109 may compare (e.g,. using HMMscan) each candidate sequence resulting from step 3 (satisfying the first confidence threshold, e.g., 0.8) to each of the models stored in the database in step 3(c), to find and eliminate sequences that have a higher confidence score (given the tolerance parameter) for any function other than the desired function. FIG. 13H shows a snippet of an example output file of filtering clustered hits against other Hidden Markov Models representing a varied array of reaction activities. In this example, the Model Identifiers represent KEGG orthology groups that represent a particular reaction activity. For each identified sequence, the figure shows the expectation-value with which the sequence matches to the HMMs in the scanning database of different activities. The expectation score of the identified sequence to the desired activity (tyrosine decarboxylase shown as TYDC_training) in relation to those of other activities quantifies how specific is the sequence to the desired activity. For example, for the sequence Q7XHL3, the desired tyrosine decarboxylase activity is not the activity with the least e-value, and hence, may not be the best candidate sequence to test.

A user-defined tolerance parameter may be used to set a limit as to how much the confidence that a candidate sequence produces a desired function is allowed to fall below a confidence that it also produces an undesired function. The prediction engine 109 may compare the confidence that a given candidate sequence enables a desired function to the confidence levels that the candidate sequence enables any other known functions stored in a database, according to their predictive models. This tolerance parameter allows the user to address cases where a candidate sequence may be predicted to match multiple functions (represented by models) with varying degrees of confidence, and the user would like to ensure that the model representing the desired function is one of the best matches (if not the best match) for the candidate sequence. For example, this tolerance can be a ratio of the (log of the lowest e-value found when compared to the database of all models) divided by the (log of the e-value when compared to the model representing the desired function). In that instance, if the best-matching model is also the one representing the desired function, the ratio will be 1. In all other cases, the ratios lower than 1 would denote decreased confidence about the given candidate sequence having the desired function and not the function represented by the model which is the best match (e.g., the once with the lowest e-value).

Example Based on Experimental Data

Using the sequence selection process essentially as illustrated by FIG. 12, path III (i.e., all the steps except the feedback learning), between 48 and 72 candidate sequences were selected for 3 enzymatic functions of interest from a meta-genomic collection of protein sequences. In the same manner 72 candidate sequences were also selected for a small-molecule exporter function of interest. Notably, all four functions were native to the microbe in which selected sequences were tested, but were deemed of interest based on the assumption that they may be limiting for production of the target molecule or its export from the cells.

Each one of the selected protein sequences was back-translated into a coding DNA sequence, synthesized and inserted in the genome of the microbe, which was already a highly-effective industrial producer of the molecule of interest. These modified microbes were tested for the improvement in production of the specific molecule in terms of two phenotypes of interest: (1) speed of production in gram per L per hour; (2) overall substrate-to-product conversion efficiency in gram per gram. Multiple sequences representing two of the three enzymatic functions and one exporter function resulted in a statistically significant improvement of over 1% for at least one of the two phenotypes of interest. In such a highly-optimized, industrially-used microbe it would be rare to observe any change that improved one of the phenotypes without a detrimental effect on the other one. Nevertheless, multiple of the candidate sequences conferred such an improvement. To measure phenotypic improvement, each of the algorithmically-selected sequences was engineered individually into the host microbe, and then the resulting phenotypic improvement was evaluated.

This experiment demonstrated utility of the workflow illustrated by FIG. 12 for finding highly efficacious candidate sequences for enzymatic and exporter functions even from a large meta-genome that consists of only predicted protein sequences without any functional annotations. The improvements in this example were obtained without the feedback learning of embodiments of the disclosure. Thus, one would expect feedback learning to result in prediction of sequences with even greater improvement.

Machine Learning

Embodiments of the disclosure may apply machine learning (“ML”) techniques to learn the relationship between given parameters (sequences) and observed outcomes (e.g., functions). In this framework, embodiments may use standard ML models, e.g. Decision Trees, to determine feature importance. In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes exhibited by training data. The result of the learning is then used to predict whether new data will exhibit the same patterns, categories, statistical relationships or other attributes.

Embodiments of this disclosure may employ unsupervised machine learning. Alternatively, some embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) or Tensor processing units (TPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

Computer System Implementation

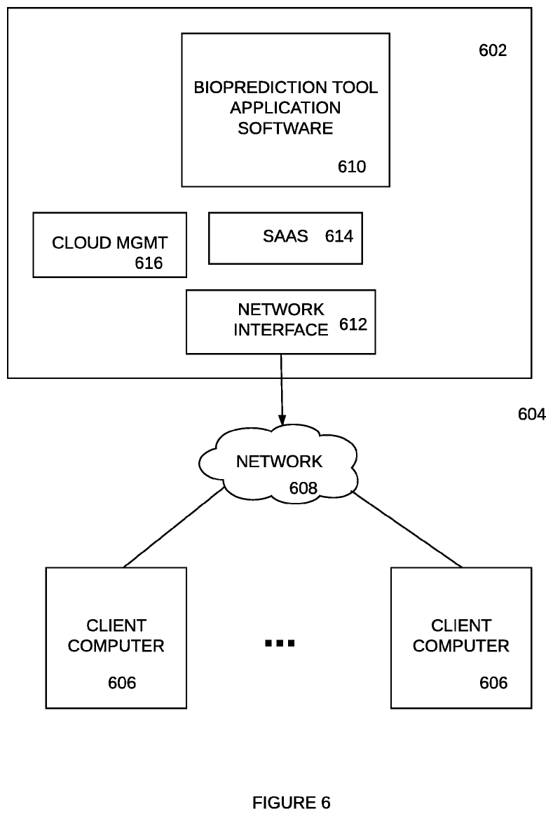

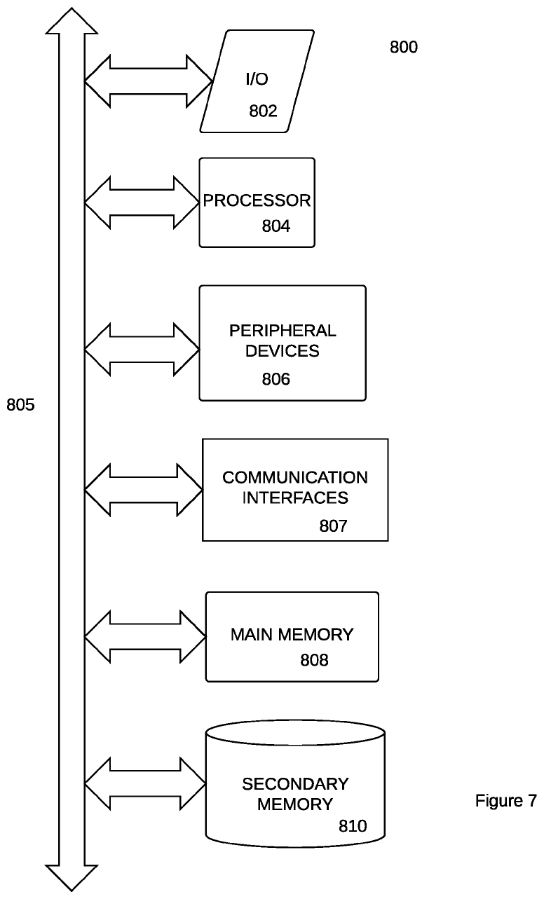

FIG. 6 illustrates a cloud computing environment 604 according to embodiments of the present disclosure. In embodiments of the disclosure, the software 610 for the reaction annotation engine 107 and the prediction engine 109 of FIG. 1 may be implemented in a cloud computing system 602, e.g., to enable multiple users to annotate reactions and predict bioreachable molecules according to embodiments of the present disclosure. Client computers 606, such as those illustrated in FIG. 7, access the system via a network 608, such as the Internet. The system may employ one or more computing systems using one or more processors, of the type illustrated in FIG. 7. The cloud computing system itself includes a network interface 612 to interface the bioreachable prediction tool software 610 to the client computers 606 via the network 608. The network interface 612 may include an application programming interface (API) to enable client applications at the client computers 606 to access the system software 610. In particular, through the API, client computers 606 may access the annotation engine 107 and the prediction engine 109.

A software as a service (SaaS) software module 614 offers the bioreachable prediction tool system software 610 as a service to the client computers 606. A cloud management module 616 manages access to the system 610 by the client computers 606. The cloud management module 616 may enable a cloud architecture that employs multitenant applications, virtualization or other architectures known in the art to serve multiple users.

FIG. 7 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the annotation engine 107 and the prediction engine 109, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, the elements of bioreachable prediction tool and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 6.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they are disclose essential matter.

In the claims below, a claim n reciting “any one of the preceding claims starting with claim x,” shall refer to any one of the claims starting with claim x and ending with the immediately preceding claim (claim n-1). For example, claim 35 reciting “The system of any one of the preceding claims starting with claim 28” refers to the system of any one of claims 28-34.

1. A computer implemented method for identifying a candidate biological sequence for enabling a function in a host cell, the method comprising:
a) predicting, using a predictive model that associates a plurality of biological sequences with one or more functions, that one or more candidate sequences of the plurality of biological sequences enable a desired function;
b) classifying, using a processor, candidate sequences that satisfy a confidence threshold as filtered candidate sequences,
i) wherein processing of one or more first filtered candidate sequences of the filtered candidate sequences would result in production of one or more corresponding molecules; and
c) returning data representing the filtered candidate sequences.

2.-25. (canceled)

26. A system for identifying a candidate biological sequence for enabling a function in a host cell, the system comprising:
one or more processors; and
one or more memories storing instructions, that when executed by at least one of the one or more processors, cause the system to:
a. predict, using a predictive model that associates a plurality of biological sequences with one or more functions, that one or more candidate sequences of the plurality of biological sequences enable a desired function;
b. classify using a processor, candidate sequences that satisfy a confidence threshold as filtered candidate sequences,
i. wherein processing of one or more first filtered candidate sequences of the filtered candidate sequences would result in production of one or more corresponding molecules; and
c. return data representing the filtered candidate sequences.

27.-50. (canceled)

51. One or more non-transitory computer-readable media storing instructions for identifying a candidate biological sequence for enabling a function in a host cell, wherein the instructions, when executed by one or more computing devices, cause at least one of the one or more computing devices to:
a. predict, using a predictive model that associates a plurality of biological sequences with one or more functions, that one or more candidate sequences of the plurality of biological sequences enable a desired function;
b. classify candidate sequences that satisfy a confidence threshold as filtered candidate sequences,
i. wherein processing of a first filtered candidate sequence of the filtered candidate sequences would result in production of a molecule; and
c. return data representing the filtered candidate sequences.

52. The one or more non-transitory computer-readable media of claim 51, storing instructions that, when executed, cause at least one of the one or more computing devices to:
a. obtain empirical data concerning whether at least one of the filtered candidate sequences enables the desired function; and
b. refine the predictive model using the empirical data.

53. The one or more non-transitory computer-readable media of claim 51, wherein the predictive model employs machine learning.

54. The one or more non-transitory computer-readable media of claim 51, wherein classifying comprises classifying a diversified set of the candidate sequences that satisfy the confidence threshold as the filtered candidate sequences.

55. The one or more non-transitory computer-readable media of claim 54, wherein classifying the diversified set as the filtered candidate sequences comprises:
a. clustering, into each cluster of a plurality of clusters, a plurality of candidate sequences that satisfy the confidence threshold; and
b. identifying, as included within the diversified set, at least one candidate sequence from each of at least two clusters of the plurality of clusters.

56. The one or more non-transitory computer-readable media of claim 51, wherein classifying further comprises:
a. not classifying, as a filtered candidate sequence, a candidate sequence that satisfies the confidence threshold but that is more likely to enable a function different from the desired function.

57. The one or more non-transitory computer-readable media of claim 56, wherein not classifying comprises not classifying, as a filtered candidate sequence, a candidate sequence that satisfies the confidence threshold but that is more likely, within a given tolerance, to enable a function different from the desired function.

58. The one or more non-transitory computer-readable media of claim 51, wherein the biological sequences are enzyme amino acid sequences, and the desired function is an enzyme-catalyzed reaction.

59. The one or more non-transitory computer-readable media of claim 51, wherein the biological sequences include enzyme amino acid sequences and the one or more enzymatic functions are one or more enzyme-catalyzed reactions along one or more reaction pathways, each reaction pathway for producing a molecule.

60. The one or more non-transitory computer-readable media of claim 51, wherein the biological sequences include nucleotide sequences that code for enzymes, and the desired function is an enzyme-catalyzed reaction.

61. The one or more non-transitory computer-readable media of claim 51, wherein processing comprises engineering into the host cell at least one nucleotide sequence corresponding to at least one of the one or more first filtered candidate sequences.

62. The one or more non-transitory computer-readable media of claim 51, wherein the predictive model is based at least in part upon sequence alignment.

63. The one or more non-transitory computer-readable media of claim 51, wherein the predictive model is based at least in part upon at least one of the following models: Hidden Markov Model (HMM), artificial neural network, or dynamic Bayesian network.

64. The one or more non-transitory computer-readable media of claim 51 storing instructions that, when executed, cause at least one of the one or more computing devices to provide to a gene manufacturing system information concerning the one or more first filtered candidate sequences, wherein the gene manufacturing system is operable to enable the host cell to use the one or more first filtered candidate sequences to enable a reaction pathway to produce the one or more molecules.

65. The one or more non-transitory computer-readable media of claim 51 storing instructions that, when executed, cause at least one of the one or more computing devices to cause production of at least one of the one or more molecules using at least one of the one or more first filtered candidate sequences.

66. The one or more non-transitory computer-readable media of claim 51, wherein the one or more molecules are bioreachable molecules.

67. The one or more non-transitory computer-readable media of claim 51, wherein the function is one of a transcription function or a transport function.

68. The one or more non-transitory computer-readable media of claim 51, wherein the one or more molecules are one or more of the filtered candidate sequences.

69. The one or more non-transitory computer-readable media of claim 51, wherein one of the filtered candidate sequences comprises an enzyme amino acid sequence, the molecule is a bioreachable molecule, and processing comprises catalyzing a reaction using the enzyme amino acid sequence.

70. The one or more non-transitory computer-readable media of claim 51, wherein the one or more molecules include molecule one or more molecules predicted to be one or more bioreachable molecules.

71. The one or more non-transitory computer-readable media of claim 51, wherein the one or more molecules are predicted by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites for the host cell and metabolites generated in previous processing steps, to generate data representing the one or more molecules.

72. The one or more non-transitory computer-readable media of claim 71, wherein selecting comprises selecting reactions that are indicated as catalyzed by one or more corresponding catalysts that are themselves indicated as able to be engineered into an organism or taken up from the growth medium in which an organism is grown.

73. The one or more non-transitory computer-readable media of claim 71, wherein selecting comprises selecting reactions that are indicated as catalyzed by one or more corresponding catalysts that are themselves indicated as corresponding to one or more amino acid sequences or one or more genetic sequences.

74. The one or more non-transitory computer-readable media of claim 71, wherein selecting comprises selecting reactions based at least in part upon whether the reactions are indicated in at least one database as catalyzed by one or more corresponding catalysts that are themselves indicated as available to catalyze the reactions.

75. The one or more non-transitory computer-readable media of claim 51, wherein the host cell originates from a microbe, a plant, or animal tissue, or is part of a single-celled organism or a multi-celled organism.

76. One or more non-transitory computer-readable media storing instructions for identifying a candidate biological sequence for enabling a function in a host cell, wherein the instructions, when executed by one or more computing devices, cause at least one of the one or more computing devices to:
access data representing filtered candidate sequences, wherein the filtered candidate sequences are determined by:
a. predicting, using a predictive model that associates a plurality of biological sequences with one or more functions, that one or more candidate sequences of the plurality of biological sequences enable a desired function; and
b. classifying candidate sequences that satisfy a confidence threshold as filtered candidate sequences, wherein processing of a first filtered candidate sequence of the filtered candidate sequences would result in production of a molecule.
